(19) United States
(12) Patent Application Publication
(10) Pub. No.: US 2023/0398286 A1
(43) Pub. Date: Dec. 14, 2023

(54) SYSTEMS AND METHODS FOR SUBSTANTIALLY CONTINUOUS INTRAVENOUS INFUSION OF THE SAME OR SUBSTANTIALLY THE SAME MEDICAL FLUID WITH FLUID SOURCE REPLACEMENTS

(71) Applicant: ICU Medical, Inc., San Clemente, CA (US)

(72) Inventors: James D. Jacobson, Lindenhurst, IL (US); Gerald W. Brann, Antioch, IL (US); Kerin L. Klagges-Kingsbury, Boxford, MA (US); Robert P. Cousineau, Boston, MA (US)

(21) Appl. No.: 18/329,375

(22) Filed: Jun. 5, 2023

Related U.S. Application Data
(60) Provisional application No. 63/351,747, filed on Jun. 13, 2022.

(51) Int. Cl. A61M 5/142; A61M 5/168

(52) U.S. Cl. CPC A61M 5/14216; A61M 5/16854; A61M 5/16831; A61M 2005/14208; A61M 2005/16863

(57) ABSTRACT

Disclosed in some embodiments is an electronic intravenous infusion pump provided with a disposable, insertable pump cartridge that is connectable to one or more intravenous fluid infusion sources, wherein the pump is coupled to a first fluid reservoir and a second fluid reservoir, wherein fluid is selectively drawn from the first fluid reservoir and the second fluid reservoir to provide substantially continuous infusion of substantially the same medical fluid to a patient.

### BACKGROUND

### Field

This disclosure relates to intravenous infusion pumps, including electronically controlled intravenous infusion pumps.

### Related Art

Patients all over the world who are in need of medical care commonly receive intravenous infusion therapy, especially during surgery or when hospitalized. This process generally involves accessing the patient's veinous system via a needle or catheter, often placed in the hand or arm, and then coupling the needle or catheter to a tubing set in communication with one or more different types of therapeutic fluids. Once connected, the fluid travels from the fluid source(s), through the tubing set and catheter, and into the patient. The fluid can provide certain desired benefits to the patient, such as maintaining hydration or nourishment, diminishing infection, reducing pain, lowering the risk of blood clots, maintaining blood pressure, providing chemotherapy, and/or delivering any other suitable drug or other therapeutic liquid to the patient. Electronic infusion pumps in communication with the fluid sources and the patient can help to increase the accuracy and consistency of fluid delivery to patients, but current electronic infusion pumps present opportunities for further improvement.

### SUMMARY

In some implementations, an electronic intravenous infusion pump is provided with a disposable, insertable pump cartridge that has at least two fluid inlets that are selectively connectable to two or more intravenous fluid infusion sources and/or supply lines, respectively. The pump can be configured to sequentially draw liquid beginning with an initial one of the fluid inlets until the intravenous fluid infusion source in communication with that fluid inlet is depleted, then transfer automatically to a different fluid inlet until the respective intravenous fluid infusion source in communication with that different inlet is depleted, and then again transfer automatically to yet another inlet or back to the initial inlet, and so on. The cycle is repeatable continuously by automatically transferring to draw liquid from a fluid source or supply line that is not empty, that is full, that contains liquid, or that has been replenished. In some implementations, a healthcare provider does not need to be present at the precise moment when a particular intravenous fluid infusion source becomes depleted to switch the fluid flow to another source or to replace or fill the depleted intravenous fluid infusion at that moment. Rather, the healthcare provider can set up a substantially continuous flow of intravenous fluid by programming the pump and then periodically replacing depleted intravenous fluid infusion sources or supply lines at a convenient time in his or her workflow. If air is introduced into a fluid line by a depleted fluid infusion source (e.g., an IV bag), the pump can be configured to sense the air and reverse the liquid flow to return the air into the depleted bag or into a new supply container without producing a clinically significant interruption in patient infusion. Air can also or alternatively be removed by trapping it in a disposable cassette.

In some implementations a control system for controlling operation of an infusion pump of an infusion pump system is provided. The infusion pump system can include a first fluid reservoir and a first supply line, a second fluid reservoir and a second supply line, a disposable cassette with an interior common channel in selective fluidic communication with the first fluid reservoir and the second fluid reservoir, and an infusion pump. The common channel can be in fluid communication with an outlet tube that is in fluid communication with a patient's venous system. The infusion pump is operable to drive fluid through the common channel into a patient delivery line. The system includes one or more hardware processors. The system includes a memory storing executable instructions that when executed by the one or more hardware processors, configure the infusion pump to: draw fluid from the first fluid reservoir through the common channel; automatically discontinue drawing fluid from the first fluid reservoir and begin drawing fluid from the second fluid reservoir through the common channel upon receiving an indication that the first fluid reservoir is depleted; and automatically discontinue drawing fluid from the second fluid reservoir and begin drawing fluid from the replaced or replenished first fluid reservoir through the common channel upon receiving an indication that the second fluid reservoir is depleted. Fluid drawn from the first fluid reservoir and delivered to the patient through the common channel can be substantially successively continuous with fluid drawn from the second fluid reservoir and delivered to the patient through the common channel. Further, incremental, sequential replacement or replenishment of the reservoirs could continue for many cycles, as necessary.

In some implementations, a method for controlling operation of an infusion pump of an infusion pump system is provided. The infusion pump system includes a first fluid reservoir, a second fluid reservoir, a common channel in selective fluidic communication with the first fluid reservoir and the second fluid reservoir, and an infusion pump. The infusion pump is operable to drive fluid through the common channel. The method includes drawing fluid from the first fluid reservoir through the common channel; automatically discontinuing drawing fluid from the first fluid reservoir and drawing fluid from the second fluid reservoir through the common channel upon receiving an indication the first fluid reservoir is depleted; and automatically discontinuing drawing fluid from the second fluid reservoir and drawing fluid from the replaced or replenished first fluid reservoir through the common channel upon receiving an indication that the second fluid reservoir is depleted. Fluid drawn from the first fluid reservoir through the common channel is substantially successively continuous with fluid drawn from the second fluid reservoir through the common channel. Further, incremental, sequential replacement or replenishment of the reservoirs could continue for many cycles, as necessary.

In some implementations a control system for controlling operation of an infusion pump of an infusion pump system is provided. The infusion pump system includes a first fluid reservoir, a second fluid reservoir, a common channel in selective fluidic communication with the first fluid reservoir and the second fluid reservoir (such as through the action of one or more valves), and an infusion pump. The infusion pump is operable to drive fluid through the common channel. The control system includes one or more hardware processors and a memory storing executable instructions that when executed by the one or more hardware processors, configure the infusion pump to: draw fluid from the first fluid reservoir through the common channel; automatically discontinue drawing fluid from the first fluid reservoir and draw fluid from the second fluid reservoir through the common channel upon receiving an indication that the first fluid reservoir is depleted; and automatically discontinue drawing fluid from the second fluid reservoir and draw fluid from the replaced or replenished first fluid reservoir through the common channel upon receiving instructions to draw fluid from the first fluid reservoir. Fluid drawn from the first fluid reservoir through the common channel is substantially successively continuous with fluid drawn from the second fluid reservoir through the common channel. Fluid delivered to the patient is substantially successively continuous across these transitions as well. Further, incremental, sequential replacement or replenishment of the reservoirs could continue for many cycles, as necessary.

In some implementations, a method for controlling operation of an infusion pump of an infusion pump system is provided. The infusion pump system includes a first fluid reservoir, a second fluid reservoir, a common channel in selective fluidic communication with the first fluid reservoir and the second fluid reservoir, and an infusion pump. The infusion pump is operable to drive fluid through the common channel. The method includes drawing fluid from the first fluid reservoir through the common channel; automatically discontinuing drawing fluid from the first fluid reservoir and drawing fluid from the second fluid reservoir through the common channel upon receiving an indication that fluid is depleted from the first fluid reservoir; and automatically discontinuing drawing fluid from the second fluid reservoir and drawing fluid from the replaced or replenished first fluid reservoir through the common channel upon receiving instructions to draw fluid from the first fluid reservoir. Fluid drawn from the first fluid reservoir and delivered to the patient through the common channel can be substantially successively continuous with fluid drawn from the second fluid reservoir and delivered to the patient through the common channel. Further, incremental, sequential replacement or replenishment of the reservoirs could continue for many cycles, as necessary.

### BRIEF DESCRIPTION OF THE DRAWINGS

The following drawings and the associated descriptions are provided to illustrate implementations of the present disclosure and do not limit the scope of the claims.

FIGS. 1A-E show front perspective, front elevational, rear elevational, top plan, and side elevational views, respectively, of an example of an infusion pump.

FIG. 2A shows an example of a cassette that can be used with the pump of FIG. 1.

FIGS. 2B-2D shows an example of a cassette that is the same as or similar to the cassette of FIG. 2A that can be used with the pump of FIG. 1.

FIG. 2E shows an example of a cassette that is the same as or similar to the cassette of FIG. 2A that can be used to draw fluid from a plurality of syringes.

FIG. 3A illustrates components of a pump driver that can interact with the cassette(s) of FIGS. 2A-2E.

FIG. 3B illustrates a fluid path through a cassette such as one or more of those shown in FIGS. 2A-2E, such as may be controlled by the hardware of FIG. 3A.

FIG. 3C illustrates schematically how hardware (e.g., FIG. 3A) interacts with a cassette (e.g., FIGS. 2A-2E) to affect flow along a fluid path.

FIG. 3D shows an example of a schematic diagram of some functional components of a medical pump system that can be used with or instead of those illustrated or described elsewhere in this application.

FIG. 4 is a flow diagram of an example of a substantially continuous infusion process executed by the pump.

FIG. 5 is a flow diagram of an example of a substantially continuous infusion process executed by the pump.

FIG. 6a is a graph of a typical interruption in fluid flow to a patient during a change between fluid sources, such as when a first syringe fluid source is depleted and a second syringe fluid source is attached and pumping restarted.

FIG. 6b is an example of a graph of the infusion rate of the pump during a substantially continuous infusion process.

FIG. 6c is an example of a graph of the infusion rate of the pump during a substantially constant rate that includes small increases and decreases in fluid flow infusion process.

### DETAILED DESCRIPTION

This specification provides textual descriptions and illustrations of many devices, components, assemblies, and subassemblies for providing substantially continuous or “infinite” fluid infusion to a patient. Any structure, material, function, method, or step that is described and/or illustrated in one example can be used by itself or with or instead of any structure, material, function, method, or step that is described and/or illustrated in another example or used in this field. The text and drawings merely provide examples and should not be interpreted as limiting or exclusive. No feature disclosed in this application is considered critical or indispensable. The relative sizes and proportions of the components illustrated in the drawings form part of the supporting disclosure of this specification, but should not be considered to limit any claim unless recited in such claim. Fluid is a substance, such as a liquid or gas, that is capable of flowing and that changes its shape when acted upon by a moderate force. Liquid is a fluid that can be infused into a patient during intravenous therapy.

### Examples of Advantages In Some Implementations

Patients frequently receive intravenous therapy from liquid source containers in the form of IV bags or syringes that are attached to electronically controlled large volume infusion pumps which draw from the source container. As liquid is pumped out of an IV bag, the walls of the bag draw together, effectively decreasing the volume within the bag; and as liquid is pumped out of a syringe by the action of the pump, the syringe plunger advances distally, effectively decreasing the liquid containing volume within the constant volume syringe barrel. In some embodiments, when a vented syringe adaptor is used to draw from a syringe, air can replace fluid expelled from the syringe volume. In some embodiments, an electronically readable data source can be provided on the first or second fluid source container or reservoir, such as an RFID tag, a barcode, or a QR code, that can provide any or all information relevant to a patient infusion, such as one or more of a patient's identification, the name of the medication in the source container or reservoir, the concentration of the medication in the source container or reservoir, and/or the administration instructions for the medication in the source container or reservoir.

In the busy workflow of hospitals and other healthcare settings, it is not always possible for healthcare providers to be present at the moment when a patient's IV bag is depleted in order to supply a new bag and reprogram the patient's IV pump for a new course of infusion. Thus, in many instances, a patient's IV infusion is temporarily halted while a patient waits for a healthcare provider to provide a new IV bag or syringe. However, for some patients, especially those under intensive medical care, a substantially continuous flow of certain medications is needed for an extended time period. For example, a patient may require continuous delivery of a vasoactive medication to maintain blood pressure, etc. When these medical fluids or any other suitable type of substantially continuously infused medical fluid are interrupted, the level of medication in the patient's blood stream may decrease below an acceptable level, the therapeutic effect may be diminished, and the patient's progress or healing may be halted or reversed. With respect to delivery from bags, clinicians can add fluid to an existing bag as the bag is in fluid communication with the pump and patient, which introduces confusion on the total volume remaining and the expiry of the bag, now with medication added at different times. Sometimes, clinicians also can hang another bag and fluidically connect it in parallel to the first bag upstream of the pump, which introduces the same limitations of unknown remaining available volume and expected expiry of now parallel infusions, and uses upstream ports on tubing sets to enable the introduction of incremental bags.

In some implementations, a substantially continuous flow of medical fluid can be provided to a patient even during transitions between infusion sources (e.g., when switching between depleted and fluid-containing IV bags or syringes), even when air is temporarily introduced into a pumping cassette or line, and/or even without requiring that a healthcare provider be present at the instant when a fluid source is depleted of fluid. An intravenous fluid infusion pump can be programmed to substantially continuously infuse medical fluid to a patient for an open-ended or effectively “infinite” time period, including when transitioning from a first fluid source to one or more other sources of substantially the same fluid and then back to the replaced or replenished first fluid source of substantially the same fluid in a substantially continuous manner. When a cassette-based infusion pump is used to draw fluid from source containers, the fluid sources can be in fluid communication with the cassette via a plurality of distinct ports on the upstream side of the cassette. A medical fluid cassette can be a disposable component that is configured to be quickly attached and removed from a medical fluid pump. The medical fluid cassette can receive fluid in an interior space and can include components that are useful in pumping, such as a pumping interface region, one or more medical fluid connectors, one or more air vents, and/or one or more sensors or sensing regions.

In some examples, depleted fluid or a deplete fluid container (such as an IV bag) can refer to a state of fluid or a fluid container that is in a depletion zone. A depletion zone is a state in which the fluid in the first fluid reservoir, the second fluid reservoir, or a subsequent fluid reservoir, is fully depleted or nearing depletion. For example, the depletion zone can include a state in which there is no fluid remaining in a reservoir (“fully depleted”), or there is only an amount of fluid remaining in a fluid reservoir that corresponds to up to about an inner volume of a fluid line extending between and connecting the fluid reservoir with another medical device (e.g., another medical fluid line, connector, cassette, cartridge, reservoir, or container), such that the remaining fluid in the reservoir can be transferred out of the reservoir and into the other medical device without introducing air or vacuum from the reservoir (“nearing depletion”). In some examples, the depletion zone can be represented as a state in which a reservoir contains an amount of fluid remaining in a fluid reservoir that will be fully depleted, leaving the reservoir completely empty, in a specified amount of time. For example, the depletion zone can be represented as a state in which an amount of fluid remaining in a reservoir will be fully depleted at a given rate of infusion within about thirty seconds or within about one minute. In some examples, the time period can be preset in the controller or set by a user. The depletion zone can be determined in any other way as described in any place in the specification. For example, the identification of a reservoir in a fluid depletion zone can be accomplished by electronically sensing the absence of fluid or the presence of an air bubble or vacuum in one or more portions of the system, such as one or more of a medical fluid reservoir, a medical fluid line, a medical fluid cassette, and/or a medical fluid connector (e.g., a Y-connector). The electronic sensing can be accomplished using any suitable device or method, such as infrared or ultrasonic sensing.

Depletion of a fluid source can be detected or sensed in one or more ways, including: (a) sensing of a fluid pressure reduction in a fluid line or within the cassette; (b) sensing air in a fluid line or within the cassette; and/or (c) sensing an “occlusion” in a syringe fluid source or creation of vacuum during the pump stroke caused by the syringe plunger reaching the distal end of the syringe barrel (which can be confirmed by verifying within the controller that the syringe volume, infusion rate, and infusion time are within range of depletion). Sensing of pressure and/or pressure change can be accomplished in one or more ways, including by using one or more piezoelectric sensors, strain-gauge sensors, acoustic sensors, light (e.g., infrared) sensors, etc. In some embodiments, the sensing of an occlusion can be detected or confirmed by determining a change (e.g., decrease or increase) in the force or electrical current required to move the pumping actuator (e.g., plunger).

When air is detected in the cassette or in a fluid line, it can be rapidly backprimed towards or into a depleted source container or towards or into a current fluid container from which liquid is being currently drawn (such as when the depleted source container has been removed for refilling). In some embodiments, backpriming is accomplished by modifying the opening and closing of the electronically-controlled valves (e.g., closing the outlet valve and opening one inlet valve during the pumping stroke and then, if necessary, opening one other inlet valve during the intake stroke) until the air is removed or purged from the cassette and/or fluid line. Infusion can immediately continue thereafter without significant interruption to the flow of therapeutic fluid to the patient. One or more depleted medical fluid containers can be temporarily detached, removed, and replaced, and/or refilled conveniently at any time during a healthcare provider's workflow while substantially the same medical fluid is being simultaneously infused into the patient from another fluid source container that is also attached to the infusion pump.

In some implementations, the electronic recording and tracking of total fluid infusion into a patient can be precise and comprehensive. Rather than maintaining a separate, discrete record or log of each bag or syringe of IV fluid infused into the patient, requiring a healthcare provider to add up all of the separate bag volumes of the same type of medical fluid to determine a total infused amount, the pump can be configured to record and/or display a continuously increasing amount of a single medical fluid or combination of medical fluids that has been infused into a particular patient over a particular time period.

In some implementations, the overall cost and waste relating to infusing medical fluid into a patient can be diminished. Some medical fluids are very expensive and are provided in large containers and small containers. In some situations, the volume of the large container is several times the volume of the small container. For a particular patient, it may not be necessary to infuse all of the medication provided in a large container; rather, the patient may need only a quantity of medication that could be provided through multiple administrations of small containers that together would be less than one large container. However, in some situations, busy healthcare providers recognize that they may not always be immediately available to replace a small container of medication when depleted with another small container of medication, which could cause an undesirable discontinuity in the patient's fluid administration. Therefore, they often simply attach a large container to the infusion pump but program it to infuse only a partial amount of the fluid and then discard the remaining fluid, thereby increasing the cost and waste of fluid administration. By permitting a healthcare provider to pre-attach multiple small volumes to the infusion pump and then configure the pump to automatically transition from one to the other, the healthcare provider does not need to be present at the precise moment during the transition and can administer only the amount needed by the patient, thereby saving money, decreasing waste, enabling automatic source transitions while the clinician is not in the patient's room, and avoiding discontinuities in the medical fluid supply to a patient.

Syringe pumps that controllably force fluid out of a syringe as opposed to drawing from the syringe as an aspect of a pump filling cycle, often have long start-up times to reach programmed target rates. This is due to the pump's absorption of mechanical slack as well as to pressurize the compliant syringe and consumable tubing set before achieving accurate delivery. This start up delay is particularly pronounced when the traditional syringe pump is programmed at low and very rates, for example less than a few mL/hr or less than 1 mL/hr. In some embodiments, a pump that draws from successive syringes diminishes the delay in delivery that would otherwise be introduced each time the syringe is changed on a traditional syringe pump.

### Examples of Pump Systems

In some implementations, a pump system can include a reusable pump driver and a disposable temporary fluid holder, such as a fluid cassette, syringe, section of tubing, etc. A disposable cassette, which is typically adapted to be used only once for a single patient and/or only for limited time, is usually a small unit with a plastic housing having at least one inlet and an outlet respectively connected through flexible tubing to the fluid supply container and intravenously through a needle to the patient receiving the fluid. In some implementations, the cassette can include a pumping chamber. The flow of fluid through the chamber can be controlled by electronically actuated valves and a plunger or pumping element activated in a controlled manner by the pump driver. For example, the cassette chamber can have one wall formed by a flexible diaphragm or membrane against which the plunger is repeatedly pressed in a reciprocating manner, which causes the fluid to flow. The pump driver can include the plunger or pumping element for controlling the flow of fluid into and out of the pumping chamber in the cassette, and it may also include one or more controls and/or electronically actuated valves to help deliver the fluid to the patient at a pre-set rate, in a pre-determined manner, for a particular pre-selected time, and/or at a pre-selected total dosage.

In some implementations, during an intake pumping stroke, a first electronically controlled inlet valve can be opened and a second electronically controlled outlet valve can be closed. At the beginning of this stroke, the pump plunger and diaphragm or membrane begin in an inwardly displaced position inside of the pumping chamber. The pump plunger then withdraws from the pumping chamber, allowing the diaphragm or membrane to quickly retract or pull back from its prior inwardly displaced position to a resting position outside of the interior of the pumping chamber, effectively increasing the volume of the pumping chamber. This action draws fluid from the fluid source through the open inlet and into the pumping chamber. During a pumping stroke, the first electronically controlled inlet valve can be closed and the second electronically controlled outlet valve can be opened. The pump plunger then moves in the opposite direction, forcing the diaphragm or membrane back into the pumping chamber to advance the fluid contained in the pump chamber out through the outlet valve. By repeating this valving and pumping action in an electronically controlled manner, the fluid is urged into and out of the cassette in a series of pulses. When the pulses occur in rapid succession, the flow to the patient approximates a continuous flow.

In some embodiments, the intake stroke is very rapid (e.g., occurring over less than or equal to about 1 or about 5 seconds) but the pumping stroke is much slower (e.g., occurring over at least about 1 or about 2 or more minutes, or even extended over as long as about 2 or about 3 or more hours). The pumping stroke can be accomplished over many very small inwardly advancing steps by the pump plunger (e.g., at least about 100 steps or at least about 150 steps or at least about 500 or more steps). Although the fluid flow to the patient is interrupted intermittently for very short periods during the intake stroke, the overall fluid flow from the fluid source to the patient is substantially continuous. The interruptions in fluid flow can be of such short duration that they do not create clinically significant delays in fluid delivery to the patient. For example, the short interruptions do not normally lead to any clinically significant lowering of medication concentration in the patient's bloodstream because the time required to metabolize significant amounts of medication by a patient is much longer than the length of the individual interruptions.

Controlled pumping of fluid through a cassette can be accomplished in many ways. An example of methods and structures for pumping fluid through a cassette is disclosed in U.S. Pat. No. 7,258,534, which is incorporated by reference herein, for all that it contains, including but not limited to examples of pump drivers and disposable fluid holders. It is contemplated that any structure, material, function, method, or step that is described and/or illustrated in the '534 patent can be used with or instead of any structure, material, function, method, or step that is described and/or illustrated in the text or drawings of this specification.

### Examples of Pump System Components

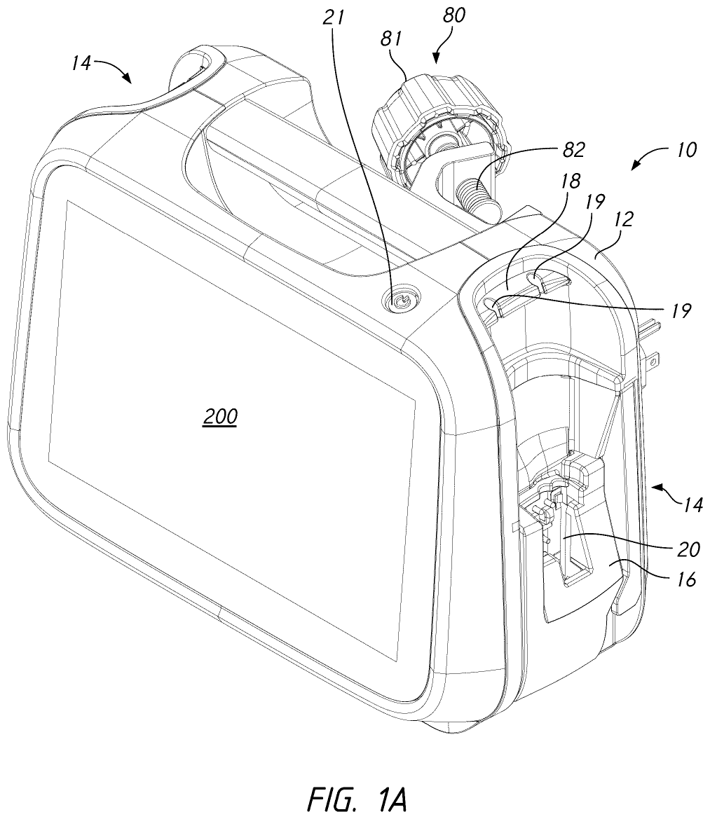

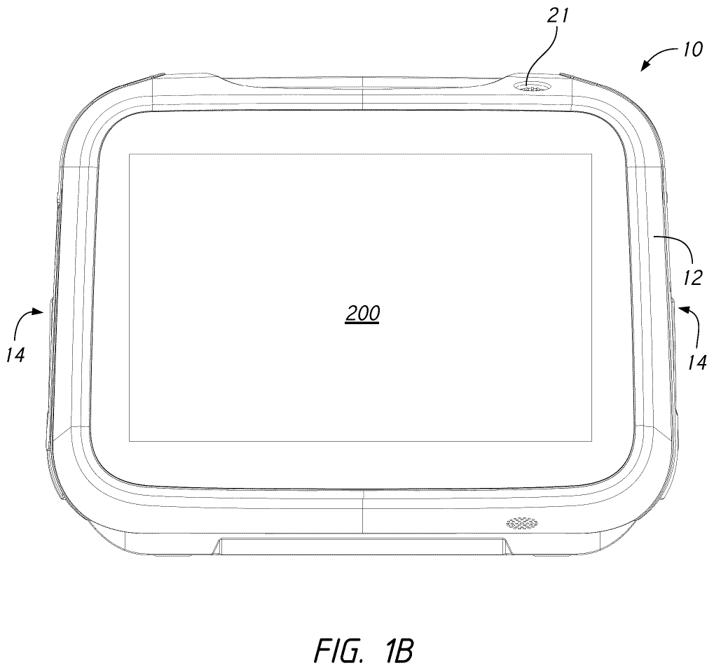

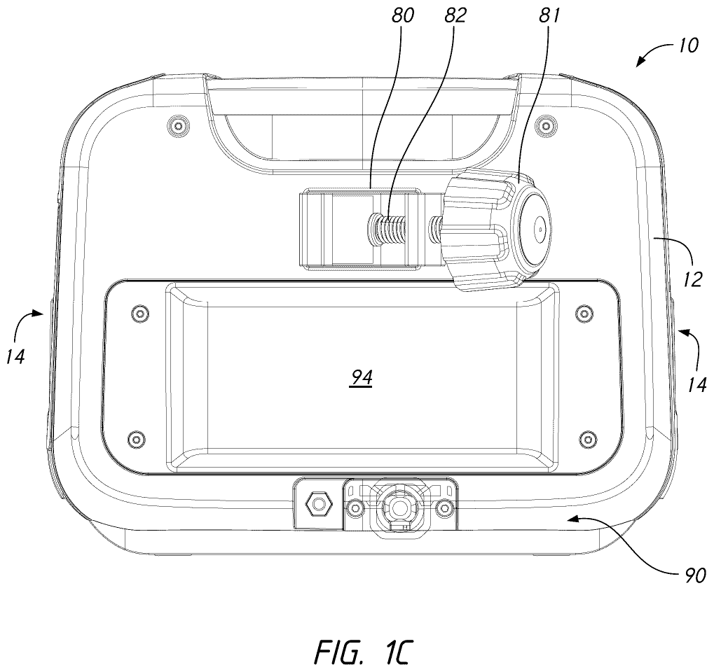

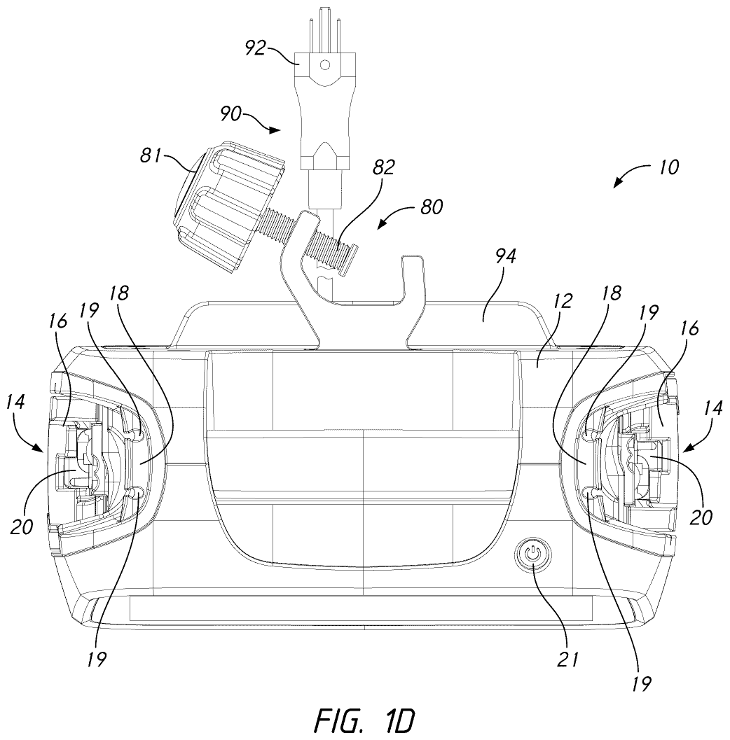

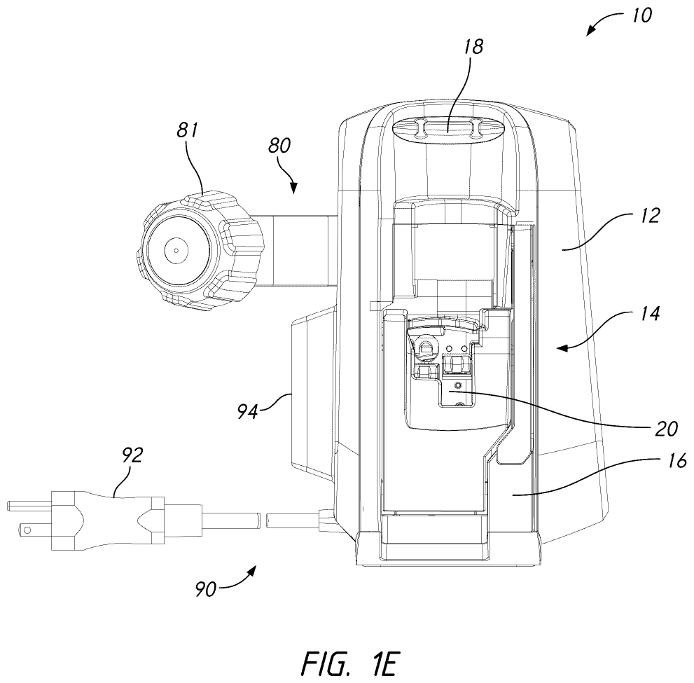

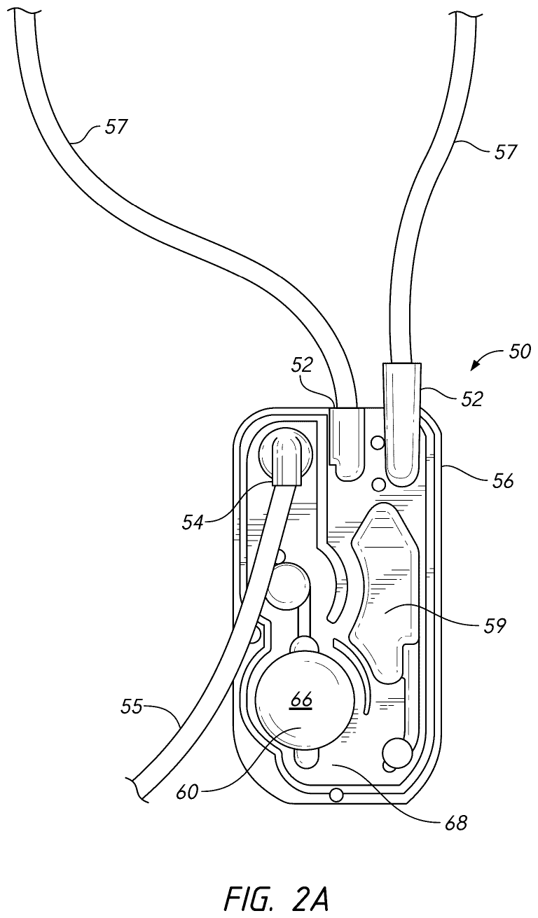

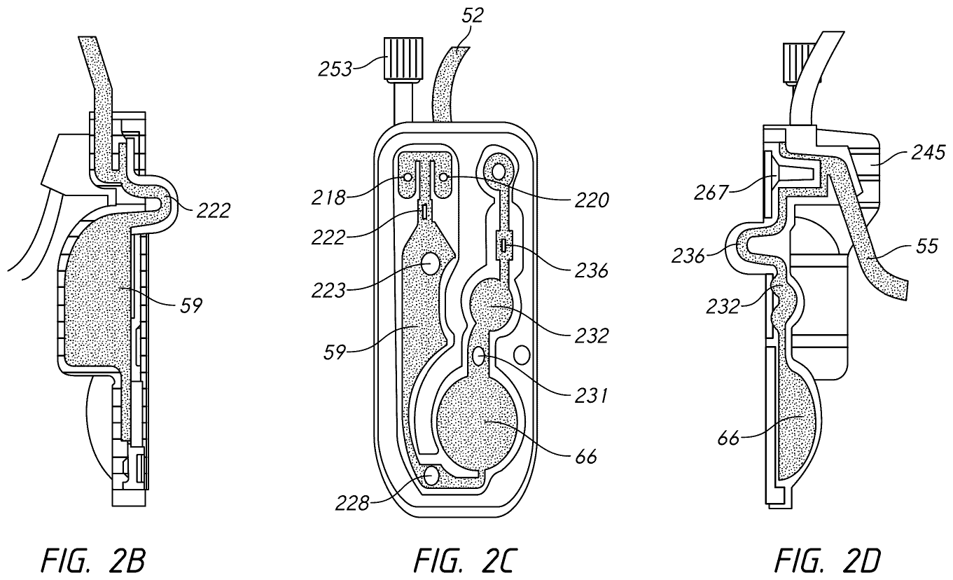

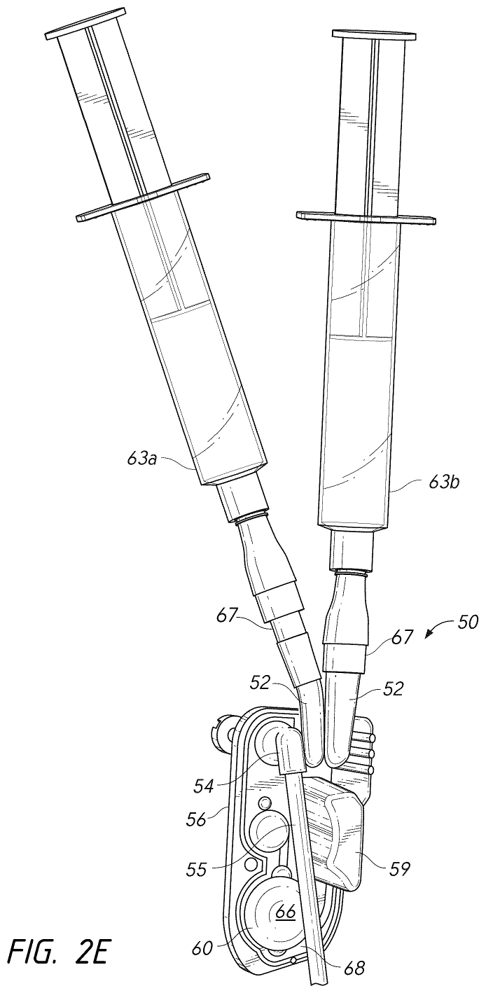

FIGS. 1A-1E show an electronic medical intravenous pump 10 with a housing 12 and at least one electromechanical pump driver 14 attached to the housing 12. As illustrated, a plurality of pump drivers 14 (e.g., at least two) can be integrally provided within the same housing 12 of a single medical pump 10. Either or both of the pump drivers 14 can include a cover 16 that partially or entirely encloses an outer surface of the pump driver 14, an indicator 18 (e.g., an illuminating communicator) attached to the cover 16, one or more tube holders 19, and a loader 20 configured to securely receive and releasably hold a disposable fluid holder (see, e.g., FIGS. 2A-2D), including but not limited to a cassette, syringe, and/or tubing. The one or more tube holders 19 can be configured to removably receive and securely hold one or more fluid-conveying tubes extending into or exiting from fluid holder when the fluid holder is received into the loader 20. The indicator 18 can communicate one or more messages to a user, such as by temporarily illuminating in one or more colors. Examples of one or more messages include confirming that a pump driver 14 near the indicator is currently active and pumping or that one or more instructions being received from a user will apply to a pump driver 14 near the indicator 18. The loader 20 can be a mechanism with multiple moving parts that opens, closes, expands, contracts, clasps, grasps, releases, and/or couples with the fluid holder to securely hold the fluid holder on or within the pump 10 during fluid pumping into the patient. The loader 20 can be integrated into and positioned on or within the pump 10 near the cover 16 adjacent to the indicator 18.

A user communicator, such as display/input device 200, can be provided to convey information to and/or receive information from a user (e.g., in an interactive manner). As illustrated, the user communicator is a touch screen that is configured to provide information to a user through an illuminated dynamic display and is configured to sense a user's touch to make selections and/or to allow the user to input instructions or data. For example, the display-input device 200 can permit the user to input and see confirmation of the infusion rate, the volume of fluid to be infused (VTBI), the type of drug being infused, the name of the patient, and/or any other useful information. The display-input device 200 can be configured to display one or more pumping parameters on a continuing basis, such as the name of the drug being infused, the infusion rate, the volume that has been infused and/or the volume remaining to be infused, and/or the elapsed time of infusion and/or the time remaining for the programmed course of infusion, etc. As shown, the touch screen can be very large, for example at least about 4 inches×at least about 6 inches, or at least about 6 inches×at least about 8 inches. In the illustrated example, the touch screen fills substantially the entire front surface of the pump 10 (see FIG. 1A), with only a small protective boundary surrounding the touch screen on the front surface. As shown, the touch screen comprises at least about 80% or at least about 90% of the surface area of the front of the pump 10. In some implementations, the front of the touch screen comprises a clear glass or plastic plate that can be attached to the housing 20 in a manner that resists liquid ingress, such as using a water-proof gasket and/or adhesive that can withstand repeated exposure to cleaning and sanitizing agents commonly used in hospitals without significant degradation.

An actuator 21 can be provided separate from the user communicator. The actuator 21 can be configured to receive an input and/or display information to a user. As shown, the actuator 21 is a power button that permits the user to press on the actuator 21 to power up the pump 10. The actuator 21 can illuminated to communicate to the user that the pump 10 is power on. If the power source is running low, the actuator 21 can change the color of illumination to quickly show to a user that a power source needs to be replenished.

In some implementations, the user communicator, such as a display/input device 200, can alternatively or additionally comprise one or more screens, speakers, lights, haptic vibrators, electronic numerical and/or alphabetic read-outs, keyboards, physical or virtual buttons, capacitive touch sensors, microphones, and/or cameras, etc.

During use, the pump 10 is typically positioned near the patient who is receiving fluid infusion from the pump 10, usually lying in a bed or sitting in a chair. In some implementations, the pump 10 may be configured to be an ambulatory pump, which will typically include a smaller housing, user communicator, battery, etc., so as to be conveniently transportable on or near a mobile patient. In many implementations, the pump 10 is attached to an IV pole stand (not shown) adjacent to the patient's bed or chair. As shown, the pump 10 can include a connector 80 that is configured to removably attach the pump 10 to the IV pole stand. As shown, the connector 80 can comprise an adjustable clamp with a large, easily graspable user actuator, such as a rotatable knob 81, that can be configured to selectively advance or retract a threaded shaft 82. At an end of the shaft 82 opposite from the knob 81 is a pole-contacting surface that can be rotatably advanced by the user to exert a force against a selected region of the pole, tightly pushing the pole against a rear surface of the pump 10, thereby securely holding the pump 10 in place on the pole during use. The selected region of the pole where the contacting surface of the shaft 82 is coupled can be chosen so as to position the pump 10 at a desired height for convenient and effective pumping and interaction with the patient and user.

The pump 10 can include a power source 90. In some implementations, the power source can comprise one or more channels for selectively supplying power to the pump 10. For example, as illustrated, the power source 90 can comprise an electrical cable 92 configured to be attached to an electrical outlet and/or a portable, rechargeable battery 94. One or more components of the pump 10 can operate using either or both sources of electrical power. The electrical cable 92 can be configured to supply electrical power to the pump 10 and/or supply electrical power to the battery 94 to recharge or to maintain electrical power in the battery 94.

Inside of the housing 20 of the pump 10, various electrical systems can be provided to control and regulate the pumping of medical fluid by the pump 10 into the patient and/or to communicate with the user and/or one or more other entities. For example, the pump can include a circuit board that includes a user interface controller (UIC) configured to control and interact with a user interface, such as a graphical user interface, that can be displayed on the user communicator or display/input device 200. The pump 10 can include a printed circuit board that includes a pump motor controller (PMC) that controls one or more pump drivers 14. In some implementations, the PMC is located on a separate circuit board from the UIC and/or the PMC is independent from and separately operable from the UIC, each of the PMC and UIC including different electronic processors capable of concurrent and independent operation. In some implementations, there are at least two PMC's provided, a separate and independent one for each pump driver 14, capable of concurrent and independent operation from each other. The pump 10 can include a printed circuit board that includes a communications engine (CE) that controls electronic communications between the pump 10 and other entities (aside from the user), such as electronic, wired or wireless, communication with a separate or remote user, a server, a hospital electronic medical records system, a remote healthcare provider, a router, another pump, a mobile electronic device, a near field communication (NFC) device such as a radio-frequency identification (RFID) device, and/or a central computer controlling and/or monitoring multiple pumps 10, etc. The CE can include or can be in electronic communication with an electronic transmitter, receiver, and/or transceiver capable of transmitting and/or receiving electronic information by wire or wirelessly (e.g., by Wi-Fi, Bluetooth, cellular signal, etc.). In some implementations, the CE is located on a separate circuit board from either or both of the UIC and/or the PMC(s), and/or the CE is independent from and separately operable from either or both of the UIC and/or the PMC(s), each of the PMC(s), UIC, and CE including different electronic processors capable of concurrent and independent operation. In some implementations, any, some, or all of the UIC, CE, and PMC(s) are capable of operational isolation from any, some, or all of the others such that it or they can turn off, stop working, encounter an error or enter a failure mode, and/or reset, without operationally affecting and/or without detrimentally affecting the operation of any, some, or all of the others. In such an operationally isolated configuration, any, some, or all of the UIC, CE, and PMC(s) can still be in periodic or continuous data transfer or communication with any, some, or all of the others. The UIC, PMC(s), and/or CE can be configured within the housing 20 of the pump 10 to be in electronic communication with each other, transmitting data and/or instructions between or among each of them as needed.

FIG. 2A shows an example of a disposable fluid holder, such as a disposable cassette 50, that includes a plastic housing and a flexible, elastomeric silicon membrane. Any structure, material, function, method, or step that is described and/or illustrated in U.S. Pat. No. 4,842,584, which is incorporated herein by reference in its entirety, including but not limited to the pumping cassette, can be used by itself or with or instead of any structure, material, function, method, or step that is described and/or illustrated in this specification. The plastic housing of the cassette 50 can include one or more (e.g., two as shown) fluid inlets 52 and a fluid outlet 54 formed in a main body 56. The cassette 50 can be temporarily positioned for example in the loader 20 of a pump driver 14. The one or more fluid inlets 52 are coupled with one or more inlet tubes 57 in fluid communication with one or more sources of medical fluid, such as one or more IV bags, vials, and/or syringes, etc., containing medical fluid. If multiple inlets 52 and inlet tubes 57 are provided, as shown, then multiple sources of medical fluid can be simultaneously supplied to a patient through the cassette 50. The fluid outlet 54 is coupled to an outlet tube 55 in fluid communication with the patient, normally by way of a needle leading into a patient's blood vessel.

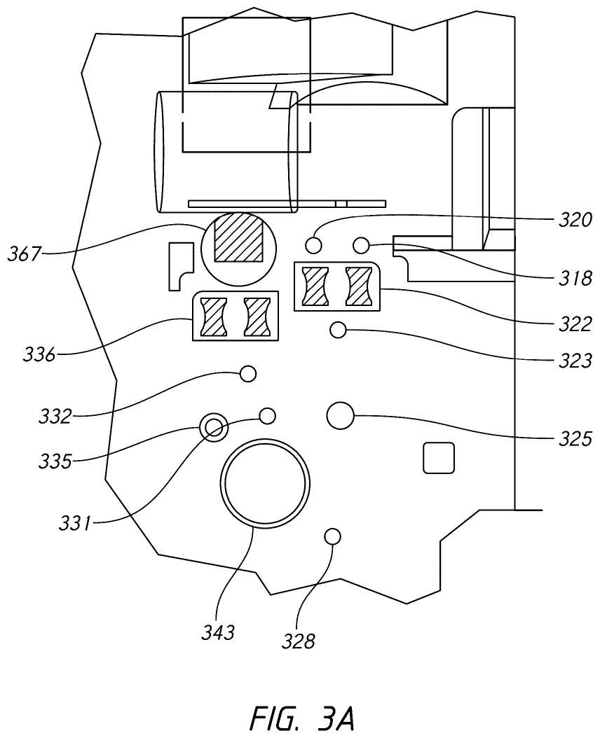

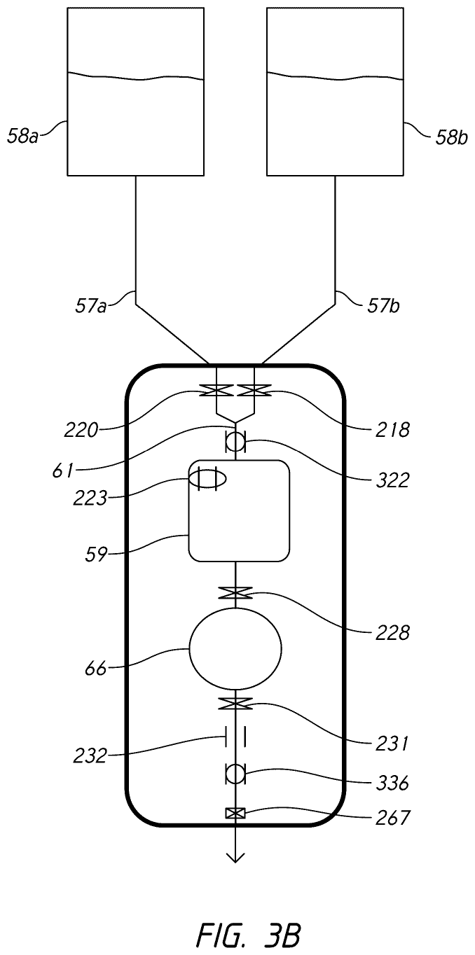

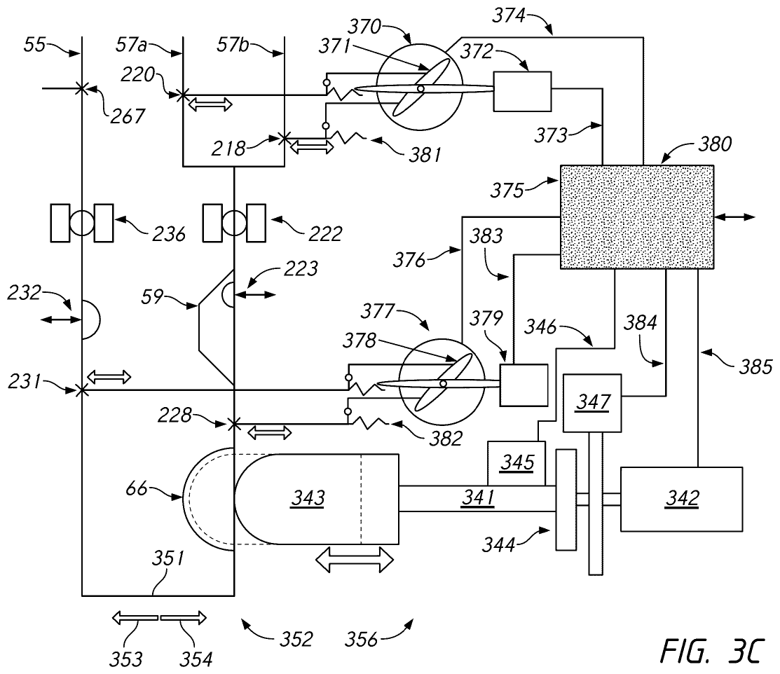

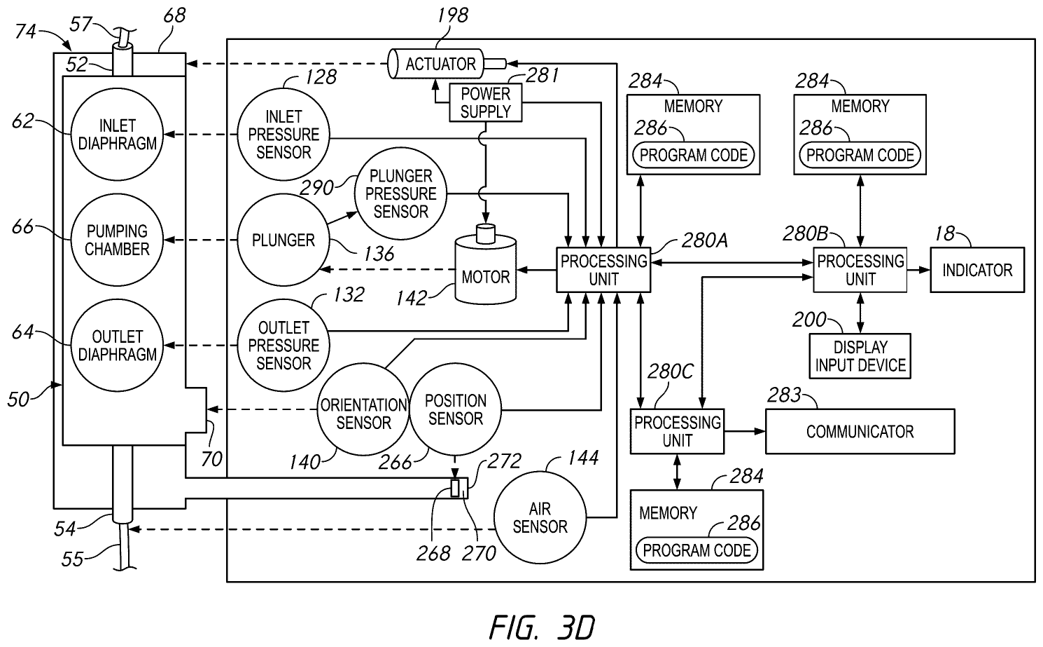

A flexible, elastomeric membrane forms a diaphragm 60 within a pumping chamber 66 on an inner face 68 of the main body 56. In operation, fluid enters through one or more of the inlets 52 and is forced through the outlet 54 under pressure. One or more fluid channels within the main body 56 of the cassette 50 convey the fluid between the inlets 52 and the outlet 54 by way of the pumping chamber 66. Before use, the cassette is typically primed with fluid, usually saline solution. A volume of fluid is delivered to the outlet 54 when a plunger 136 of the pump 10 (see, e.g., FIG. 3) displaces the diaphragm to expel the fluid from the pumping chamber 66. During an intake stroke, the plunger 136 retracts from the diaphragm 60, and the fluid is then drawn in through the inlet 52 and into the pumping chamber 66. In a pumping stroke, the pump 10 displaces the diaphragm 60 of the pumping chamber 66 to force the fluid contained therein through the outlet 54. In some implementations, the directional movement of flow can be facilitated by one or more supply line selection valve(s) (e.g., at one or more of inlet 52 or outlet 54). For example, the supply line selection valve(s) can initially be configured and controlled to direct fluid from a first fluid reservoir 58a (e.g., bag or syringe) into the common channel 61. At a later time, the supply line selection valves can be configured and controlled to switch to directing fluid from a second reservoir 58b into the common channel 61 instead of from the first fluid reservoir 58a. The fluid can flow from the cassette 50 in a series of spaced-apart pulses rather than in a continuous flow. In some implementations, the pump 10 can deliver fluid to a recipient (e.g., a patient) at a pre-set rate, in a pre-determined manner, and for a particular (e.g., pre-selected) time or total dosage. The cassette 50 can include an air trap 59 in communication with an air vent (not shown).

FIGS. 2B, 2C, and 2D show three views of a cassette that is the same as or similar to the cassette of FIG. 2A. In FIGS. 2B and 2C, fluid can flow into an inlet 52, from a primary container, for example. Fluid can also flow into a secondary port 253, which can have a Y-connector with a resealable opening or a locking cap. Fluid coming in from the inlet 52 can pass through an A valve 220. Fluid coming in through a secondary port 253 can pass through a B valve 218. Fluid coming in through these two valves can then pass by a proximal air-in-line sensor 222. Fluid can then pass by, in a widening passage, a proximal pressure sensor 223.

FIG. 2E shows an example of a cassette that is the same as or similar to the cassette of FIG. 2A coupled to syringes 63a 63b. The inlets 52 are each coupled to resealable needle-free medical connectors 67 known as the Microclave connectors sold by ICU Medical, Inc. in San Clemente, California. Each of the needle-free medical connectors 67 are disposed between and coupled to one of the syringes 63a, 63b.

### Cassette Air Trap

The widened passage can form an air trap chamber 59, which can allow for fluid mixing. The air trap chamber is also shown in the side view of FIG. 2B. The air trap chamber 59 can be integral to the cassette. The air trap can be exposed to view above the upper edge of the cassette door when the door is closed. Air passes the proximal air-in-line sensor 222 before entering the air trap, which in some implementations can have a volume of at least about 2.0 mL (e.g., 2.15 mL). The proximal pressure sensor (see, e.g., pressure sensor 223 of FIG. 3C) can monitor pressure in the air trap chamber 59. In some implementations, the user can remove air or fluid from the proximal tubing and cassette air trap after the cassette door is closed. To remove air in the trap or the proximal tubing the user may be required to attach a container to a Line B port (e.g., secondary port 253 of FIG. 2C). A key, button, or other control (e.g., on an infuser display screen) can be selected to backprime when a delivery is not in progress. When the user selects backprime, for example, this can initiate rapid pumping of fluid from Line A to a user-attached container on Line B. In some implementations, no fluid is delivered to the cassette distal line during a backprime. After the backprime control is released, a cassette leak test can be automatically performed.

In some implementations, after passing through an air trap chamber 59, fluid can subsequently flow through an inlet valve 228 and from there into a pumping chamber 66. The pumping chamber 66 is also shown in the side view of FIG. 2D. From the pumping chamber 66, fluid can flow through an outlet valve 231 and then into a widened passage accessed by a distal pressure sensor 232. This passage subsequently narrows down to pass a distal air-in-line sensor 236. The two air-in-line sensors, proximal 222, and distal 236, can both be positioned near a bend in a passage or tubing, as shown in the side views of FIGS. 2B and 2D. Fluid can flow through or pass a precision gravity flow regulator 267, seen in FIG. 2D. A finger grip 245 is also seen protruding to the right in FIG. 2D. An outlet tube 55 is also shown coming from the precision gravity flow regulator 267 and leading to a patient. The features shown in the cross-sectional schematics of FIGS. 2B-2D can correspond generally to the external cassette contours shown in FIG. 2A.

### Fluid Delivery

A pumping system or infuser can deliver fluids from two or more fluid sources through a sterile fluid pathway of administration set tubing, accessories, and a cassette. In some implementations, there is no contact between the fluid and an infusion mechanism subsystem (see FIG. 3A and the electromechanical portion 356 of FIG. 3C). In some implementations, the pumping force can be provided by one or more of the structures, configurations, processes, and/or control systems shown in FIGS. 2A through 3D, but many other additions or alternatives can also be used, including a peristaltic or a syringe pump with suitable valving and valve controls of the type disclosed in one or more implementations in this specification to help accomplish substantially continuous infusion.

In some implementations, a pumping system can be programmed or set up by a user to enter a multi-step therapy program to perform an infusion of the same or substantially the same medical fluid in a substantially continuous manner by automatically sequentially delivering fluid from a first line and then from one or more additional lines and then returning to the first line. Fluid flow to the patient is still considered to be substantially continuous even though short interruptions in patient fluid flow may occur during the fluid intake stroke of pumping, or during automatic transitions between one line and another after fluid source depletion is detected, or during air or bubble purging steps. Substantially continuous fluid flow can include short, discrete, and/or predictable interruptions in fluid flow that do not lead to clinically significant decreases in infused fluid volume or medication concentration in a patient's bloodstream. For example, in some situations, the automatic switching of fluid source containers can occur in less than or equal to about 10 seconds, while the “half life” of medication concentration in the bloodstream is much longer, such as at least about 2 minutes, and in most cases much longer than that.

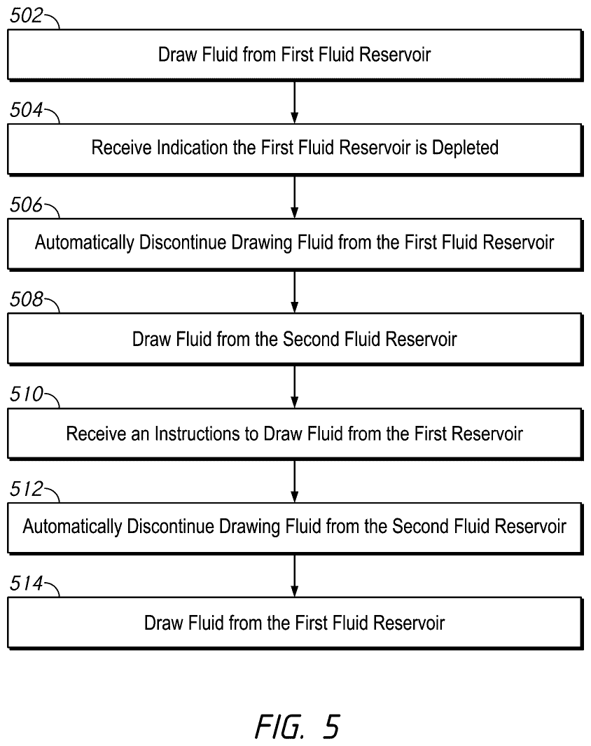

An additional or alternative infusion pump cassette that can be used with any implementation in this specification is illustrated in FIG. 5 of U.S. Pat. No. 7,402,154. An elastomeric membrane 60 forms an inlet diaphragm 62, an outlet diaphragm generally indicated at 64, and a pumping chamber 66 located between the inlet and outlet diaphragms 62 and 64 on an inner face 68 of the main body 56. In operation, fluid enters through the inlet 52 and is forced through outlet 54 under pressure. The fluid is delivered to the outlet 54 when the plunger 136 of the pump 10 displaces the pumping chamber 66 to expel the fluid. During the intake stroke the plunger 136 releases the pumping chamber 66, and the fluid is then drawn through the inlet 52 and into the pumping chamber 66. In a pumping stroke, the pump 10 displaces the pumping chamber 66 to force the fluid contained therein through the outlet 54. The directional movement of flow can be facilitated by one or more supply line selection valve(s) (e.g., at one or more of inlet 52 or outlet 54). At low rates the flow can be delivered in discrete volumes as the pump 10 displaces the pump chamber in successive steps. Thus, the fluid can flow from the cassette 50 in a series of spaced-apart pulses rather than in a smoothly continuous flow. Typically, this pump can deliver fluid to a recipient (e.g., a patient) at a pre-set rate, in a pre-determined manner, and for a particular (e.g., pre-selected) time or total dosage. A flow stop can be formed as a switch in a main body and protrude from the inner surface 68. This protrusion can form an irregular portion of the inner surface 68 which can be used to align the cassette 50 as well as monitor the orientation of the cassette 50. The flow stop can provide a manual switch for closing and opening the cassette 50 to fluid flow. A rim 72 is located around the outer surface of the main body 56 and adjacent the inner surface 68. The rim 72 can be used to secure the cassette in a fixed position relative to the pump 10 of U.S. Pat. No. 7,402,154.

FIG. 3A illustrates an example of hardware or components of the pump driver 14 that can be configured to interact with a fluid holder such as the cassette of FIGS. 2A-2D. In FIG. 3A, an A valve interface 320 can correspond to or interact with an A valve 220. Similarly, a B valve interface 318 can correspond to or interact with a B valve 218 as shown in FIG. 2C. A proximal air-in-line sensor 322 can be located outside of a cartridge and can interact with a loop or bend in at least partially transparent fluid pathway, for example. In the illustrated example, the sensor 322 is depicted with two vertical portions that can pinch or otherwise be positioned adjacent to a tube running vertically between them. A proximal pressure sensor interface 323 can interact with a pressure sensor 223. A force-sensor, such as resistor 325, can be used to determine whether a cartridge is in physical contact with the hardware, or a portion of a pump having the hardware, shown in FIG. 3A. In some implementations, an inlet valve 228 is actively driven and can receive actuation from an inlet valve interface 328. Similarly, an outlet valve interface 331 can interact with an outlet valve 231. A plunger 343 can extend toward and interact with a pumping chamber 66 (see FIGS. 2C and 2D). A cassette locator 335 can be used to provide alignment and registration of physical interacting components when a cassette such as shown in FIGS. 2A-2D is inserted into or aligned with the hardware components shown in FIG. 3A. A distal pressure sensor interface 332 is located below a distal air-in-line sensor 336. Above this is located a regulator actuator 367, which can be configured to interact with the precision gravity flow regulator 267.

FIG. 3B illustrates a fluid path from the first fluid reservoir 58a and the second fluid reservoir 58b through a common channel 61 of a cassette such as the fluid path shown in the cassette(s) of FIGS. 2A-2D, as actuated by the hardware of FIG. 3A. The physical components of FIGS. 2A-2D and FIG. 3A can control and evaluate fluid in the path illustrated in FIG. 3B. In FIG. 3B, fluid coming in from either a primary line 57A or a secondary line 57B can pass through the A valve 220 or the B valve 218, respectively. The medical fluid can pass by a proximal in line air sensor 322 in the common channel 61 to permit a processor in the pump to detect whether there are air bubbles or vacuum space in the fluid and/or whether a fluid source has been depleted. In some situations where the A valve 220 and the B valve 218 are rapidly opening and closing, the incoming fluid can merge and/or mix in the common channel 61. However, when the cassette is used for substantially continuous fluid infusion of the same or substantially the same medical fluid, the fluid coming in from both the primary line 57A and the secondary line 57B is the same or substantially the same. In a first phase, the pump is configured so that the A valve 220 is opened and the B valve 218 is closed until the air sensor 322 and processor detect that fluid coming from the primary line 57A is depleted, at which point in a second phase the A valve is closed and the B valve 218 is opened until the air sensor 322 and processor detect that the fluid coming from the secondary line 57B is depleted, at which point the pump returns to the first phase in which A valve 220 is opened again and the B valve 218 is closed again. While the B valve 218 is open and fluid is pumping from the secondary line 57B, the healthcare provider can replace the depleted fluid source attached to the primary line 57A with a new container of substantially the same fluid (e.g., a new IV bag) and/or can refill the depleted fluid source attached to the primary line 57A with substantially the same fluid. Similarly, while the A valve 220 is open and fluid is pumping from the primary line 57A, the healthcare provide can replace the depleted fluid source attached to the second line 57B with a new container of substantially the same fluid (e.g., a new IV bag) and/or can refill the depleted fluid source attached to the secondary line 57B with substantially the same fluid. This pattern or cycle of automatic pump and valve control, and fluid source replacement by the healthcare provider, can continue indefinitely until stopped by the healthcare provider or until an error occurs (e.g., when a depleted bag is not replaced before the pump begins drawing from that bag).

After passing through the common channel 61 within the cassette, the medical fluid can then enter an air trap chamber 59 having a proximal pressure sensor 223. From here, fluid can flow through an inlet valve 228 and from there into a pumping chamber 66. From the pumping chamber 66, fluid can flow through an outlet valve 231, past a distal pressure sensor 232, and past a distal air-in-line sensor 336. Fluid can flow through or pass a precision gravity flow regulator 267 before proceeding from a cassette toward a patient through tubing.

In a system using active, positively-controlled valves with motors, during fluid delivery, the plunger (e.g., 343 in FIGS. 3A and 3C) can repeatedly cycle between the home position and the extended position. To draw fluid into the pumping chamber (e.g., 66) the inlet valve (e.g., 228) is opened. The outlet valve can then promptly close. In some implementations, opening of the inlet valve can automatically cause the outlet valve (e.g., 231) to close. When the plunger reaches the home position, the plunger motion pauses while the inlet valve (e.g., 228) is closed, pressure is equalized, and the outlet valve (e.g., 231) is opened. Then the plunger extends and the positive pressure forces fluid out of the pumping chamber and into the distal line (e.g., 55) of the set, which can be connected to a patient.

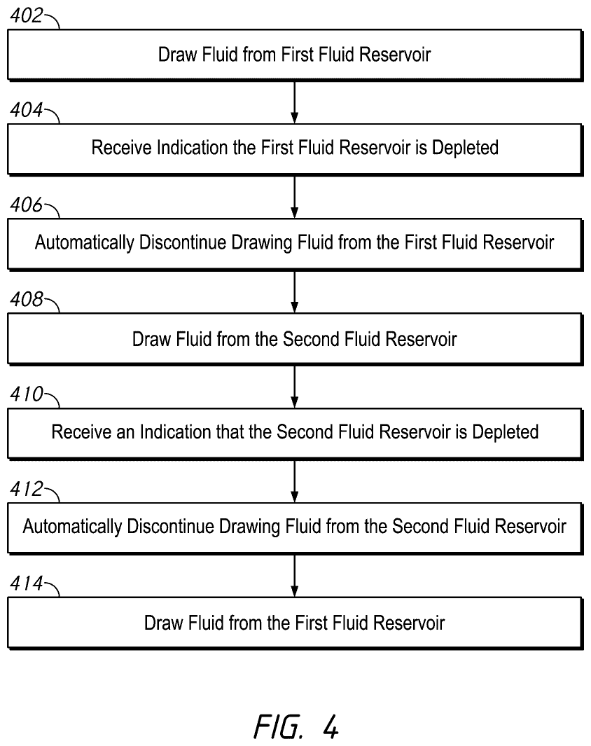

The plunger stepper motor (e.g., motor 342 of FIG. 3C or the motor of FIG. 4C) can be activated by current pulses through the motor windings. In some implementations, a plunger motor can use different patterns (e.g., 6 different patterns) of pulses can be used, depending on the delivery rate. As the rate increases, a pause between successive steps of the motor decreases. In some implementations, valve motors can use a single pattern of current pulses through the motor windings. The patterns of current pulses for the motors are advantageously controlled by a PMC microcontroller (e.g., in the controller 380).

FIG. 3C further illustrates schematically how hardware (e.g., FIG. 3A) can interact with a cassette (e.g., FIGS. 2A-2D) along a fluid path. FIG. 3C shows a patient or distal line 55 at the top left corner. At the left is shown an example of a consumable or cassette portion 352. At the right is shown an example of an electromechanical portion 356. In the cassette 352, a distal side 353 is toward the left, and a proximal side 354 is toward the right. A fluid path 351 is illustrated, passing generally from inlets 57A and 57B to outlet 55. Line A 57A leads to a Line A valve or pin 220, which can move to the right and left as shown by the arrow. Similarly, Line B 57B can lead to a Line B valve or pin 218. A spring such as the spring 381 can be deployed with respect to both the valve 218 and the valve 220, and a cam 371 can connect a stepper motor 370 with the valve to 220 and the valve 218. The stepper motor 370 can interact with a line AB position sensor 372, with feedback 373 provided to a controller or controllers 380. A controller 380 can in turn provide input and/or power 374 to the stepper motor 370. In this arrangement, the valves 220 and 218 are actively and positively controlled by a motor and a controller.

For the outlet valve and pin 231 and the inlet valve and pin 228, a stepper motor 377 having a cam 378 and associated springs 382 can interact with the valves 228 and 231. In some implementations, the cam 371 can cause the associated valves 220, 218 not to be opened simultaneously. In some implementations, the inlet valves 220 and 218 are not open simultaneously so that fluid does not mix in either of inlet lines 57A or 57B.

Similarly for the cam 378 and the valves 231 and 228, if the cam forms a rigid elongate structure as shown, it can pull on one valve while pushing on the other and when it swings the other direction push and pull in an alternating manner. The valves 228 and 231 can open at alternating times such that fluid intake occurs during a draw portion of a plunger stroke, and fluid is expelled during a push portion of a plunger stroke. Having the valves open simultaneously or other synchronization problems can be avoided to discourage backflow.

An input-output valve position sensor 379 can be connected to a physical component of the stepper motor 377. The sensor 379 can provide feedback to the controller or controllers 380, which can in turn send input and/or power 376 to the stepper motor 377.

The controller or controllers 380 can also interact with a third stepper motor 342, which can cause movement of a lead screw 341 connected to a plunger or piston 343, which in turn physically interacts with the pumping chamber 66. A linear position sensor 345 can provide feedback 346 of this process to a controller 380. Similarly, a rotary position sensor 347 can provide feedback 384 to a controller 380. Thus, linear and rotary position feedback can be provided either as a backup, as an alternative, or otherwise. A coupler 344 can be provided between the stepper motor of 342 and the lead screw 341. Input and/or power 385 can be provided from the controller 380 to the stepper motor 342. The plunger or piston 343 can follow a reciprocating pattern as shown by the arrow. Thus, the electromechanical portion 356 of a pump can have multiple reciprocating portions and multiple motors. The reciprocation of the valves 220, 218, 231 and 228 can be timed and coordinated with the reciprocation of the piston 343 (e.g., by controller/s 380) to encourage fluid to move through the fluid path 351. Although additional feedback lines are not shown in FIG. 3C, sensor feedback can be provided from the distal air inline sensor 236 and the proximal area line sensor 222, as well as the distal pressure sensor 232 and the proximal pressure sensor 223.

### Valve Operation

Valve motors such as the motors 370 and 377 of FIG. 3C can be controlled by a pump mechanism controller (“PMC”) microcontroller using a chopper motor drive. The valve motors 370 and 377 can be the same, with one motor used for a pair of valves.

An Inlet/Outlet (I/O) valve motor (e.g., 377 in FIG. 3C) opens and closes the cassette pumping chamber inlet and outlet valves (e.g., 228, 231) in an administration set cassette. The cassette can have a membrane that is exposed by openings in the back of the cassette body above where there are valve chambers in the cassette. The inlet valve pin (e.g., 228) is opened to allow fluid to enter the pumping chamber (e.g., 66) through the air trap (e.g., 59) from the proximal line, which is selected by the Line A/B Select valves (e.g., 218, 220). When the pumping chamber is filled the inlet valve (e.g., 228) is closed, the pumping chamber pressure is set and the outlet valve (e.g., 231) is opened to allow fluid to be pumped into the distal line of the set.

A state machine (e.g., in or associated with the controller 380) can run a program for controlling the I/O valve motor (e.g., 370, 377). In an optical approach, cam flags can protrude from a portion of the drive train. Rotational cam flag signals can be acquired optically during or after each motor step and are monitored using a state machine. As with the other motors, if there is an error in the Inlet/Outlet valve motor position (phase loss), then the motor can be re-initialized to the current position.

The Line A/B Select (LS) valve motor (e.g., 370 in FIG. 3C) opens and closes the Line A and Line B select valves (e.g., 220, 218) in the administration set cassette, using openings in the back of the cassette body for actuator access. The Line A valve (e.g., 220) controls the primary inlet port to the cassette which can be attached permanently to the set proximal tubing. The Line B valve (e.g., 218) controls the secondary inlet port, which may have a screw cap, a Pre-pierced or a Clave attached to it, depending on the type of set.

### Example System Operations

In some implementations, a pump system can have a cassette door with a handle that supports an administration set cassette such as that illustrated in FIGS. 2A-2D. When the door is open in a loading position the user can slide the cassette into a slot with a cassette guide spring. When the door is closed the cassette is aligned and the front of the cassette makes contact with a door datum surface, actuator and sensor subassemblies (plunger 343 and pins or valves 218, 220, 228, 231) make contact with a cassette elastomeric membrane, and a cassette guide spring can push a fluid shield against the front face of a mechanism chassis. The door can be released from the handle when it is in the loading position, allowing the door to be perpendicular to the mechanism fluid shield. This allows the user to clean the rear of the door and the fluid shield, or to remove any object which has fallen behind the door.

A cassette locator (see, e.g., 335 in FIG. 3A) can be a pin that helps align the cassette with the mechanism as the door is closed and keeps the cassette in the correct position during delivery.

The cassette can have a flow regulator valve (e.g., the precision gravity flow regulator 267, seen in FIG. 2D) distal to the pumping chamber (e.g., the chamber 66 of FIGS. 2A-3D). The flow regulator valve can be closed by the user after an administration set is primed. The proximal line can be clamped as an additional prevention of free flow. As the door is closed, an actuator connected to the door handle can automatically open the flow regulator valve after the pumping chamber outlet valve pin closes the outlet valve. The flow regulator valve can be used by the operator to control fluid flow rate when the administration set is used independently for a gravity drip infusion.

A reciprocating pumping piston/plunger (e.g., the plunger 343 of FIG. 3C) can be actuated by a motor (e.g., the motor 342). As schematically shown in FIG. 3C, a pump plunger motor and drive train can be perpendicular to a pumping chamber membrane opening on the rear of a cassette. The drive train can have location sensors that are monitored by motor control software on a PMC microcontroller (see controller 380 of FIG. 3C). The software can implement state machines which control the motor operation.

An inlet valve to the pumping chamber (e.g., the valve 228) can be actuated by a motor (e.g., the motor 377), and a drive train can extend an actuator through an opening in the rear of the cassette to reach the valve. The same motor can be used for the outlet valve, which can improve synchronization. A default position is with the inlet valve (e.g., the valve 228) closed by a spring (e.g., 382) which can apply steady pressure to a valve pin. The drive train (see generally 377, 378 and related structures) has a location sensor (e.g., 379) that is monitored by (383) motor control software on the PMC microcontroller (e.g., 380). The software implements state machines which can control the motor operation. The same description here generally applies to an outlet valve (e.g., 231), actuated by the same motor (e.g., 377).

Line A select valve (e.g., 220) for primary proximal fluid line A (e.g., 57A) and Line B select valve (e.g., 218) for fluid line B (e.g., 57B) can be actuated by a motor (e.g., 370). As described above for the valves 228 and 231, the valves 220 and 218 can be accessed by a drive train (which may include the cam 371 and springs such as 381) through openings in a cassette, driven by a motor (e.g., 370), as tracked by a location sensor (e.g., 372) and monitored (373) by software in a controller (380).

One or more proximal and distal air-in-line sensors (222, 236) can be used to detect air passage into (proximal) or out of (distal) the cassette. Both sensors can be ultrasound piezoelectric crystal transmitter/receiver pairs. Liquid in the cassette between the transmitter and receiver conducts the ultrasonic signal, while air does not. This can result in a signal change indicating a bubble in the line.

One or more proximal and distal MEMS pressure sensors (223, 232 of FIG. 3C) can be used to detect the pressure of the tubing into (proximal) or out of (distal) the cassette. Microelectromechanical systems (MEMS) pressure sensors are an integrated circuit, which have piezo electric resistors diffused into a micro-machined diaphragm to measure strain from a steel ball that extends through the top of the IC package. The steel ball is driven by a pressure pin which is in contact with the cassette membrane.

A cassette presence sensor detects that the cassette is in the door when it is closed. The sensor can be a dome switch mounted in an infusion mechanism subsystem fluid shield. The dome switch can make contact with the cassette when the cassette is correctly aligned with the fluid shield. The switch output signal can be acquired and processed by PMC microcontroller software (e.g., in controller 380).

Motor control interfaces can provide amplification of control signals output by the PMC microcontroller (e.g., the controller 380). PMC microcontroller software can compute motor winding current values which are converted to analog voltages by a digital-to-analog converter (DAC). The control voltages input to the motor control interface can cause amplifiers to drive the selected motor winding with current modulated by a chopper pulse width modulator controller. Preferably, one motor winding is active at a time.

Sensor interfaces in an infusion mechanism subsystem can convert air-in-line, pressure, and/or motor drive position sensor signals into analog voltage signals. The analog voltages are processed by an analog-to-digital converter (ADC) in the PMC microcontroller which outputs digital values. PMC microcontroller software state machines acquire and process data from the sensors.

Non-volatile memory in an infusion mechanism subsystem can be connected to the PMC microcontroller with a serial communications link (SPI bus). The non-volatile memory can be used to store calibration values for the motor drive trains and sensors during manufacturing. Additional system parameters and an alarm log are also stored by the PMC microcontroller in this memory.

Any control and/or feedback systems of this specification can be configured to generate highly specific, real-time data on how an infusion pump is operating and how fluid in a cassette is responding. This data already exists for precision operation of an infusion device, and it can be conveniently organized and stored (e.g., in a memory of the pump system itself). This data can provide highly accurate predictions of how and when medication will reach a target destination or achieve a particular level in a target destination. Thus, the sensors, controllers, cam flags, feedback software, etc. described herein is highly valuable in predicting further outcomes, patient medication status, and/or otherwise displaying information to a user.

FIG. 3D is a schematic diagram of some functional components for a medical pump (e.g., the pump 10 of FIGS. 1A-1E) that in some implementations can be configured with some modifications to be used in connection with the disposable cassette 50 (e.g., a modified version of FIGS. 2A-D) for delivering a fluid to a patient. Some of the components and/or functions illustrated and/or described in connection with FIG. 3D are alternatives or additions to those illustrated in the cassette of FIGS. 2A-3C. One or more processors or processing units 280 can be included in pump 10 that can perform various operations. The processing unit(s) 280 and all other electrical components within the pump 10 can be powered by a power supply 281, such as one or more components of power source 90 of pump 10. In some implementations, the processing unit 280a can be configured as a pump motor controller (PMC) to control the electric motor 142 being energized by the power supply 281. When energized, the electric motor 142 can cause the plunger 136 to reciprocate back and forth to periodically actuate, press inward, and/or down-stroke, causing plunger 136 to temporarily press on pumping chamber 66, driving fluid through cassette 50. The motor 142, plunger 136, sensors 128, 290, 132, 140, 266, 144 can be included in or as an integrated part of the pump driver 14 of the pump 10. In some implementations, as shown, the inlet pressure sensor 128 engages the inlet diaphragm 62 of cassette 50, and the outlet pressure sensor 132 engages the outlet diaphragm 64 of cassette 50. When retracting, moving outward, or on an up-stroke, the plunger 136 can release pressure from pumping chamber 66 and thereby draw fluid from inlet 52 into pumping chamber 66. Differential pressure within the cassette can drive the inlet opening during the pump chamber fill cycle. In some implementations of cassette 50, a flow stop 70 is formed as a pivotal switch in the main body 56 and protrudes a given height from the inner surface 68. This protrusion forms an irregular portion of the inner surface 68 which can be used in some implementations to align the cassette 50 as well as monitor the orientation of the cassette 50. In some implementations, one form of a flow stop 70 can provide a manual switch or valve for closing and opening the cassette 50 to fluid flow.

In some implementations, the processing unit 280a can control a loader 20 of the pump 10 with an electronic actuator 198 and a front carriage being energized by the power supply 281. When energized, the actuator 198 can drive the front carriage 74 between closed or open positions. The front carriage 74 in the open position can be configured to receive the cassette 50 and in the closed position can be configured to temporarily securely retain the cassette 50 until the front carriage is moved to the closed position. A position sensor 266 for the cassette 50 can be provided in the pump 10. The position sensor 266 can monitor the position of a slot 268 formed in a position plate 270. The position sensor 266 can monitor a position of an edge 272 of a position plate 270 within the pump 10. By monitoring the position of the position plate 270, the position sensor 266 can detect the overall position of the front carriage of the loader 20 and/or confirm that the cassette 50 is inserted into the loader 20 of the pump driver 14. The position sensor 266 can be a linear pixel array sensor that continuously tracks the position of the slot 268. Of course, any other devices can be used for the position sensor 266, such as an opto-tachometer sensor.

A memory 284 can communicate with the processing unit 280a and can store program code 286 and data necessary or helpful for the processing unit 280 to receive, determine, calculate, and/or output the operating conditions of pump 10. The processing unit 280a retrieves the program code 286 from memory 284 and applies it to the data received from various sensors and devices of pump 10. The memory 284 and/or program code 286 can be included within or integrally attached to (e.g., on the same circuit board) as the processing unit 280a, which in some implementations can be the configuration for any processor or processing unit 280 in this specification.

In some implementations, the program code 286 can control the pump 10 and/or track a history of pump 10 operation details (which may be recorded and/or otherwise affected or modified, e.g., in part by input from sensors such as air sensor 144, position sensor 266, orientation sensor 140, outlet pressure sensor 132, plunger pressure sensor 290, inlet pressure sensor 128, etc.) and store and/or retrieve those details in the memory 284. The program code 286 can use any one or more of these sensors to help identify or diagnose pumping problems, such as air in a pumping line, a pumping obstruction, an empty fluid source, and/or calculate expected infusate arrival time in a patient. The display/input device 200 can receive information from a user regarding a patient, one or more drugs to be infused, and details about a course of infusion into a patient. The display/input device 200 can provide a clinician with any useful information regarding the pumping therapy, such as pumping parameters (e.g., VTBI, remaining volume, infusion rate, time for infusion, elapsed time of infusion, expected infusate arrival time, and/or time for completion of infusion, etc.) Some or all of the information displayed by the display/input device 200 can be based on the operation details and calculations performed by the program code 286.

In some implementations, the operation details can include information determined by the processing unit 280a. The processing unit 280a can process the data from pump 10 to determine some or all of the following operating conditions: whether or when the cassette 50 has been inserted, whether or when the cassette 50 is correctly oriented, whether or when the cassette 50 is not fully seated to the fixed seat 162, whether or when the front carriage assembly 74 is in an open or closed position, whether or when a jam in the front carriage assembly 74 is detected, whether or when there is proper flow of fluid through the cassette 50 to the patient, and whether or when one or more air bubbles are included in the fluid entering, within, and/or leaving cassette 50. The processing unit 280a can be configured to determine one or more operating conditions to adjust the operation of the pump 10 to address or improve a detected condition. Once the operating condition has been determined, the processing unit 280a can output the operating condition to display 200, activate an indicator window, and/or use the determined operating condition to adjust operation of the pump 10.

For example, the processing unit 280a can receive data from a plunger pressure sensor 290 operatively associated with the plunger 136. The plunger pressure sensor 290 can sense the force on plunger 136 and generate a pressure signal based on this force. The plunger pressure sensor 290 can communicate with the processing unit 280a, sending the pressure signal to the processing unit 280a for use in helping to determine operating conditions of pump 10.

The processing unit 280a can receive an array of one or more items of pressure data sensed from the cassette inner surface 68 determined by the plunger pressure sensor 290 and inlet and outlet pressure sensors 128 and 132. The processing unit 280a can combine the pressure data from the plunger pressure sensor 290 with data from inlet and outlet pressure sensors 128 and 132 to provide a determination as to the correct or incorrect positioning of cassette 50. In normal operation, this array of pressure data falls within an expected range and the processing unit 280a can determine that proper cassette loading has occurred. When the cassette 50 is incorrectly oriented (e.g., backwards or upside down) or when the cassette 50 is not fully seated to the fixed seat 162, one or more parameters or data of the array of pressure data falls outside the expected range and the processing unit 280a determines that improper cassette loading has occurred.

As shown, in some implementations, the processing unit 280a can receive data from one or more air sensors 144 in communication with outlet tube 55 attached to the cassette outlet 54. An air sensor 144 can be an ultrasonic sensor configured to measure or detect air or an amount of air in or adjacent to the outlet 54 or outlet tube 55. In normal operation, this air content data falls within an expected range, and the processing unit 280a can determine that proper fluid flow is in progress. When the air content data falls outside the expected range, the processing unit 280a can determine that improper air content is being delivered to the patient.

Processing unit 280a can continuously or periodically communicate with an independent and separate processor or processing unit 280b to communicate information to the user and/or to receive data from the user that may affect pumping conditions or parameters. For example, processing unit 280a can communicate by wire or wirelessly with processing unit 280b which can be configured as a user interface processor or controller (UIC) to control the output and input of display/input device 200, including by displaying an operating condition and/or activate indicator 18 to communicate with a user. In some implementations, processing unit 280b can receive user input regarding pumping conditions or parameters, provide drug library and drug compatibility information, alert a user to a problem or a pumping condition, provide an alarm, provide a message to a user (e.g., instructing a user to check the line or attach more fluid), and/or receive and communication information that modifies or halts operation of the pump 10.

An independent and separate processor or processing unit 280c can be configured as a communications engine (CE) for the pump, a pump communications driver, a pump communications module, and/or a pump communications processor. Processing unit 280c can continuously or periodically communicate with processing units 280a and 280b to transmit and/or receive information to and from electronic sources or destinations separate from, outside of, and/or remote from, the pump 10. As shown, processing unit 280c can be in electronic communication with or include a memory 284 and program code 286, and processing unit 280c can be in communication with and control data flow to and from a communicator 283 which can be configured to communicate, wired or wirelessly, with another electronic entity that it separate from the pump 10, such as a separate or remote user, a server, a hospital electronic medical records system, a remote healthcare provider, a router, another pump, a mobile electronic device, a near field communication (NFC) device such as a radio-frequency identification (RFID) device, and/or a central computer controlling and/or monitoring multiple pumps 10, etc. The communicator 283 can be or can comprise one or more of a wire, a bus, a receiver, a transmitter, a transceiver, a modem, a codec, an antenna, a buffer, a multiplexer, a network interface, a router, and/or a hub, etc. The communicator 283 can communicate with another electronic entity in any suitable manner, such as by wire, short-range wireless protocol (Wi-Fi, Bluetooth, ZigBee, etc.), fiber optic cable, cellular data, satellite transmission, and/or any other appropriate electronic medium.

As shown schematically in FIG. 3, a pump 10 can be provided with many components to accomplish controlled pumping of medical fluid from one or more medical fluid sources to a patient. For example, one or more processors or processing units 280 can receive various data useful for the processing unit(s) 280 to calculate and output the operating conditions of pump 10. The processing unit(s) 280 can retrieve the program code 286 from memory 284 and apply it to the data received from various sensors and devices of pump 10, and generate output(s). The output(s) are used to communicate to the user by the processing unit 280b, to activate and regulate the pump driver by the processing unit 280a, and to communicate with other electronic devices using processing unit 280c.

### Substantially Continuous Infusion

In some implementations, the user can enter a therapy program that sequentially delivers fluid from a first line, then from one or more other lines, and then from the first line again. For example, the first line can be used to start delivering a first quantity of medical liquid. After fluid delivery from the first line is completed, then the second line delivery is automatically started. In some implementations, the processor 280 is configured to provide substantially continuous infusion during operation such that the pump 10 alternates between drawing fluid from the first fluid reservoir 58a and the second fluid reservoir 58b (and/or from other reservoirs) generally seamlessly and without significant interruption of fluid flow to the patient. The first fluid reservoir 58a and the second fluid reservoir 58b can be replaced and/or refilled any desired number of times without interrupting infusion to a patient. The fluid in the first fluid reservoir 58a and the fluid in the second fluid reservoir 58b can be the same or substantially the same (e.g., the same or substantially the same type of fluid and/or the same concentration of fluid), and the fluid that replaces the fluid in the first fluid reservoir 58a when depleted and the fluid in the second fluid reservoir 58b that replaces the fluid in the second reservoir 58b when depleted can be the same or substantially the same, such that a patient can receive a uniform or substantially the same supply of medical fluid when the pump 10 draws fluid from the first fluid reservoir 58a or the second fluid reservoir 58b. Providing a generally uniform, same, or substantially the same, type of fluid from the first fluid reservoir 58a and the second fluid reservoir 58b allows a healthcare provider to replenish a medical fluid supply in the first reservoir 58a or the second reservoir 58b without interrupting infusion to a patent in a clinically significant way. Providing substantially continuous infusion also allows a healthcare provider to replace one of the fluid reservoirs 58a, 58b within an extensive window of time while the pump 10 draws from the alternate fluid reservoir 58a, 58b.

In some embodiments, when a healthcare provider desires to program the pump 10 for substantially continuous or “infinite” infusion between or among multiple, successive fluid sources, a user can begin by pressing a button or series of buttons on a touchscreen or in hardware on the pump 10 to initiate the substantially continuous infusion process. The pump 10 can prompt the user to attach at least two fluid sources with the same or substantially the same fluid contents to the cassette that is inserted into the pump 10. If the healthcare provider attaches only one fluid source, the pump 10 can remind the healthcare provider to attach the second fluid source. If the healthcare provider initiates pumping before attaching the second fluid source, the pump 10 can begin pumping but also remind and allow the healthcare provider to attach the second fluid source at any time before the first fluid source is depleted, which still permits substantially continuous infusion. In some embodiments of substantially continuous fluid infusion, the healthcare provider has flexibility to set up the additional fluid source at any point over a long period of time during infusion of the existing fluid source without requiring the healthcare provider to be present at the exact instant when a fluid source is depleted.

FIG. 4 is a flow diagram showing an implementation of substantially continuous infusion using the pump 10. In the implementation shown in FIG. 4, the pump 10 provides a substantially successively continuous flow of medical fluid during a generally seamless and substantially uninterrupted transition between drawing from the first reservoir 58a to drawing from the second reservoir 58b, at which point fluid in the first fluid reservoir 58a is replaced or refilled. Upon depletion of medical fluid in the second reservoir 58b, the pump 10 then provides a generally seamless and substantially uninterrupted transition to drawing medical fluid from the first reservoir 58b while fluid in the second fluid reservoir 58b is replaced or refilled.

In the example shown in FIG. 4, the internal computer program code 286 includes steps, instructions, algorithms, and/or data configured to cause the pump 10 to draw fluid 402 from the first fluid reservoir 58a through the common channel 61 of the cassette 50. The processing unit 280a receives an indication that the first fluid reservoir 58a is depleted 404 (such as by detecting air or the absence of liquid at the air-in-line sensor 322 when the reservoir is a bag, or by monitoring upstream pressure via pressure sensor 223 when the reservoir is a syringe), and automatically discontinues 406 drawing fluid from the first fluid reservoir 58a. The processing unit 280a actuates supply line selection valves in the cassette, causing the pump to draw fluid from the second fluid reservoir 58b. The processing unit 280a receives an indication that the second fluid reservoir 58b is depleted 410 (such as by detecting air or the absence of liquid at the air-in-line sensor 322 when the reservoir is a bag, or by monitoring upstream pressure via pressure sensor 223 when the reservoir is a syringe), automatically discontinues 412 drawing fluid from the second fluid reservoir 58b, and draws 414 fluid from the first fluid reservoir 58a. The fluid in the first and second fluid reservoirs 58a, 58b can be substantially the same. In some implementations, the pump 10 continues to selectively and alternatively draw fluid from the first reservoir 58a and the second reservoir 58b until the pump receives a signal from a user to stop drawing fluid or until the pump 10 encounters an error condition (such as when a depleted reservoir has not been replaced or refilled). In some implementations, the pump 10 continues to draw fluid for a preset period of time or until a preset amount of fluid has been drawn collectively from the first fluid reservoir 58a and the second fluid reservoir 58b. In some embodiments the preset period of time or preset amount of fluid drawn can be based on a known volume in the reservoir and understood pumping rates, where reservoir volume could be entered by a clinician or obtained by the pump electronically, such as via a bar code or RFID tag on the reservoir.

To draw 402 fluid from the first fluid reservoir 58a, the processing unit 280a transmits an electrical signal to the supply line selection valves of the cassette 50, which selectively control the flow of fluid from the first fluid reservoir 58a and the second fluid reservoir 58b into the common channel 61. As such the supply line selection valves cause the common channel 61 to be in selective fluidic communication with the first fluid reservoir 58a and the second fluid reservoir 58b. The supply line selection valves as controlled by the processing unit 280a direct the fluid from the first fluid reservoir 58a through the cassette 50 to the outlet of the cassette. In some implementations, fluid enters the cassette from the first fluid reservoir 58a through one or more of the inlets 52 and is forced through the outlet 54 by the pumping mechanism. The common channel 61 within the main body 56 of the cassette 50 conveys the fluid between the inlets 52 and the outlet 54 by way of the pumping chamber 66. The volume of fluid is delivered to the outlet 54 when a plunger 136 of the pump 10 (see, e.g., FIG. 3) displaces the diaphragm to expel the fluid from the pumping chamber 66.

The processing unit 280a receives indication from at least one sensor or from user input or from internal processing or calculation, that the first fluid reservoir 58a is depleted 404. The indication can be triggered by one or more of a plurality of events. For example, the processing unit 280a can be configured to halt the flow of fluid after drawing fluid from a reservoir having a known volume at a known fluid flow rate for a predetermined period of time. In some implementations, the processing unit 280a receives an indication from a timer that the pump 10 has been drawing fluid from the first fluid reservoir 58a for a period of time sufficient to empty the first fluid reservoir 58a. Alternatively or additionally, in some implementations, the processing unit 280a receives a pressure reading from the pressure sensor 223, which can selectively be in fluidic communication with the first fluid reservoir 58a and the second fluid reservoir 58b. The pressure sensor 223 monitors the fluid pressure from an outlet of the first fluid reservoir 58a and an outlet of the second fluid reservoir 58b and transmits an electrical signal to the pump 10 indicating when fluid from the first fluid reservoir 58a is no longer providing fluid pressure through the pressure sensor 223. For example, the proximal pressure sensor 223 can provide a signal to the processing unit 280a when fluid from the first fluid reservoir 58a provides an upstream fluid pressure that is below a threshold pressure and indicating that air is present in the line and/or the fluid in the first fluid reservoir 58a is depleted. In some implementations, pressure measurement is taken by a plurality of pressure sensors. For example, in some implementations, a separate pressure sensor is used to measure pressure from the first fluid reservoir 58a and the second fluid reservoir 58b respectively. In some implementations, the air-in-line sensor 322 detects that air or a lack of fluid is present in at least a portion of the common channel 61, indicating that the reservoir from which the fluid is being drawn has been depleted.

Alternatively or additionally, the first fluid reservoir 58a may be coupled to an electronic scale that is capable of determining a weight of the first fluid reservoir 58a and sending a signal to the processing unit 280a when the weight of the fluid reservoir falls below a threshold weight, indicating that the first fluid reservoir 58a is depleted. In some implementations, the threshold weight is an estimated weight of a container of the fluid reservoir that does not contain any liquid or contains a minimal amount of liquid. Alternatively or additionally, in some implementations, a user can manually indicate that the first fluid reservoir 58a is depleted. For example, a user can interact with the GUI of the display/input device 200 to send a signal to the processing unit 280a indicating that the first fluid reservoir 58a is depleted.

To draw fluid from the second fluid reservoir 58b, the processing unit 280a transmits an electrical signal to the supply line selection valves, which selectively control the flow of fluid from the first fluid reservoir 58a and the second fluid reservoir 58b into the common channel 61. As such, the supply line selection valves cause the common channel 61 to be in selective fluidic communication with the first fluid reservoir and the second fluid reservoir. The supply line selection valves as controlled by the processing unit 280a direct the fluid from the second fluid reservoir 58a through the cassette 50 to the outlet 54 of the cassette In some implementations, fluid enters the cassette 50 from the second fluid reservoir 58a through one or more of the inlets 52 and is forced through the outlet 54 under pressure. The common channel 61 within the main body 56 of the cassette 50 conveys the fluid between the inlets 52 and the outlet 54 by way of the pumping chamber 66. The volume of fluid is delivered to the outlet 54 when a plunger 136 of the pump 10 (see, e.g., FIG. 3) displaces the diaphragm to expel the fluid from the pumping chamber 66.

The processing unit 280a can be configured to receive indication from at least one sensor or from user input or from internal processing or calculation, that the second fluid reservoir 58b is depleted 404 in one or more of the same ways as described for receiving indication that the first fluid reservoir 58 a is depleted. When the processing unit 280a receives the indication that the second fluid reservoir 58b is depleted, the processing unit 280a stops drawing fluid from the second fluid reservoir 58b and switches back to drawing fluid from the first fluid reservoir 58a as before. In some implementations, the pump 10 is configured to only draw fluid from the respective first fluid reservoir 58a and the second fluid reservoir 58b when the pump 10 receives an indication of fluid availability such as fluid pressure, threshold weight, or manual indication by a user interaction with the user interface.

The reservoir in any of these embodiments can be any suitable container, such as a bag, syringe, vial, or other rigid, semi-rigid or flexible container. In embodiments where the reservoir is a bag, a length of tubing can be provided (such as 57 in FIG. 2A, or 57A/B in FIG. 4) with upstream volume that complements the reservoir. The volume of the tubing can be significant and included in volume calculations for the reservoir. When the reservoir directly connects to the cassette, such as in the case of a syringe, the volume of fluid between the reservoir and the common line can be quite small and may not need to be included in volume calculations for the reservoir.

### Reserve Bag Example

FIG. 5 is a flow diagram showing an implementation of substantially continuous fluid flow using the pump 10. In the implementation shown in FIG. 5, the pump provides a continuous flow of fluid drawn from the first fluid reservoir 58a as a primary reservoir. The pump 10 draws fluid from the second fluid reservoir 58b as a reserve reservoir while a user is replacing the first reservoir 58a. The pump 10 resumes drawing from the first fluid reservoir 58a once the first fluid reservoir 58a is replaced. For example, as shown in FIGS. 5, the internal computer program code 286 can include steps, instructions, algorithms, and/or data configured to cause the pump 10 to draw fluid 502 from the first fluid reservoir 58a through the common channel 61 of the cassette 50. The processing unit 280a receives indication that the first fluid reservoir 58a is depleted 504, and automatically discontinues 506 drawing fluid from the first fluid reservoir 58a. The internal computer program code 286 causes 508 the pump 10 to draw fluid from the second fluid reservoir 58b. The processing unit 280a receives instructions to draw 510 fluid from the first fluid reservoir 58a again upon an indication that the first reservoir is no longer depleted. Upon receiving instructions to draw 510 fluid from the first fluid reservoir 58a, the pump 10 automatically discontinues 512 drawing fluid from the first fluid reservoir 58a and draws 514 from the second fluid reservoir 58b. In some implementations, a user such as a physician or medical technician can interact with the GUI to send instructions to the processing unit 280a, to draw fluid from the first fluid reservoir 58a once the user has replaced the depleted first fluid reservoir 58a. As such, the second fluid reservoir 58b can be used to provide infinite infusion during multiple replacements of the first bag and can be replaced while the first fluid reservoir 58a is providing a primary fluid flow to a patient.

### Replacing Reservoirs

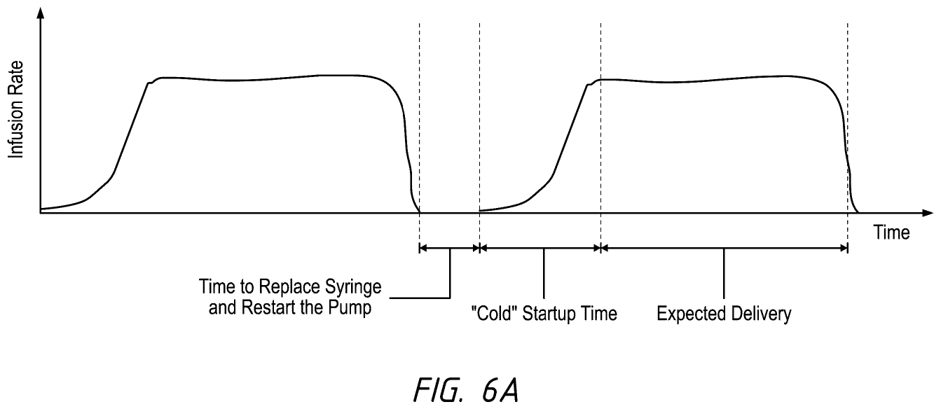

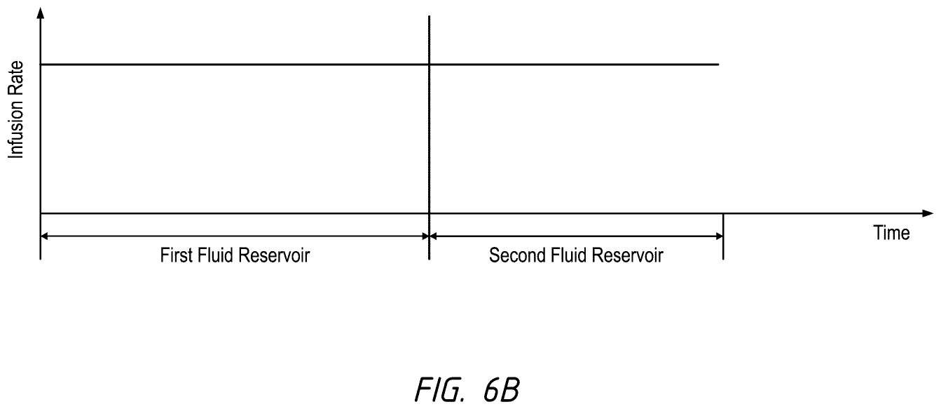

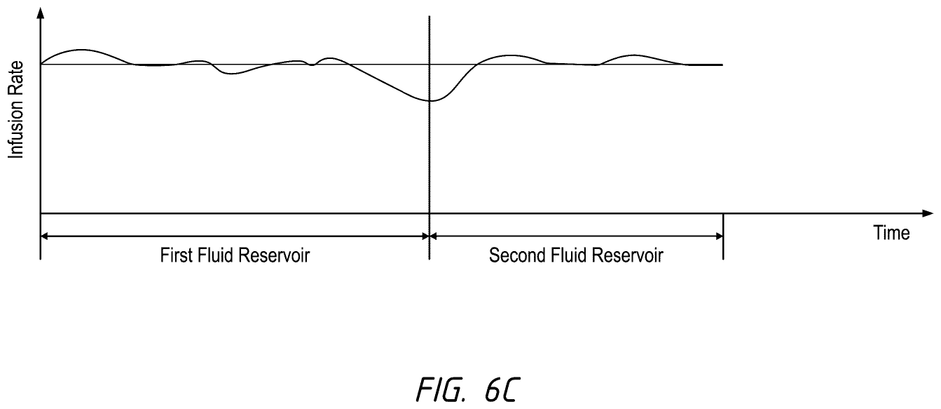

FIG. 6A shows that, during typical fluid flow from an intravenous pump, the detection of a depletion of a fluid source, the summoning of a healthcare worker to locate a replacement for and replace the depleted fluid source, and/or the attachment of a new fluid source, can introduce a significant time gap in patient infusion. In many healthcare settings, the size of the time gap is inconsistent and indeterminate because the time gap can change based on how soon a healthcare worker has the ability to replace the fluid source that is depleted. During this time gap, the fluid volume or concentration of medicine in the bloodstream of the patient may decrease significantly through natural metabolization by the patient to a point where the therapeutic effect of the IV therapy may be significantly diminished or lost. Further, when the delivery occurs via a traditional syringe pump the replacement of a depleted syringe can introduce the time delay from exchanging syringes. Additionally another time delay may arise from the syringe pump re-establishing accurate flow at the desired rate from a “cold start”.

FIG. 6B is an example infusion rate versus time diagram that shows a constant infusion rate as the pump 10 selectively draws from the first fluid reservoir 58a and then switches essentially immediately to the second fluid reservoir 58b. FIG. 6C is an example infusion rate versus time diagram that shows a more typical yet still clinically-acceptable infusion profile that can provide a substantially constant rate that can include small increases and decreases in fluid flow that are not clinically significant for a particular medication and patient, including those that occur during: (a) transitions between intake and pumping strokes (while pumping from the same source container); (b) transitions between different source containers; and/or (c) elimination or purging of air or vacuum from the pumping lines or cassette. In some embodiments of a substantially continuous infusion system, one or more of these or other short interruptions can be monitored, managed, fixed, resolved, and/or mitigated automatically by the electronic controller of the pump without any user alert and/or without any user intervention. A substantially constant rate may include intermittent interruptions and/or increases or decreases in infusion rates that are not clinically significant in view of the range of typical metabolizing rates of medications in particular categories of patients (e.g., based on age, weight, sex, drug tolerance, type of disease, injury, or other malady). For example, in some embodiments a substantially continuous infusion rate can include interruptions in flow that are consistently and predictably less than a predetermined time period that does not significantly adversely affect medication concentration in a patient, such as less than or equal to about 20 seconds, less than or equal to about 1 minute, less than or equal to about 2 minutes, or less than or equal to about 3 minutes.

In the examples shown in FIGS. 4-5, a user such as a physician can replace the first fluid reservoir 58a with a reservoir containing fluid (e.g., a full fluid reservoir) or replenish the first fluid reservoir when the first fluid reservoir 58a is determined to be empty or depleted. Fluid can be drawn by the pump from the second fluid reservoir 58b while the fluid in the first fluid reservoir 58a (or the first fluid reservoir 58a itself) is being replaced. Similarly, a healthcare provider can replace the second fluid reservoir 58b with a reservoir containing fluid (e.g., a full fluid reservoir) or replenish the second fluid reservoir when the second fluid reservoir 58b is determined to be empty or depleted and fluid is being drawn from the first fluid reservoir 58a. Each of the first fluid reservoir 58a and the second fluid reservoir 58b can be fluidically disconnected from line A and Line B respectively. A replacement first fluid reservoir 58a and second fluid reservoir 58b can be fluidically connected to Line A and Line B respectively, putting each of the first fluid reservoir 58a and the second fluid reservoir 58b in selective fluidic communication with the common channel 61. The fluid flow can be substantially continuous when the processing unit 280a activates the supply line selection valve to direct fluid from each of the respective fluid reservoirs 58a, 58b.

### Backpriming During Substantially Continuous Infusion

In some implementations, the pump 10 is configured to backprime to remove any air or any excess air that may enter Line A, Line B, or the common 61 line upon depletion of a particular reservoir or during the transition between drawing fluid from the first fluid reservoir 58a and the second fluid reservoir 58b. For example, the pump 10 can backprime during at least one instance where air or a region lacking fluid from a reservoir is drawn into the cassette and detected by a sensor upon depletion of the reservoir or while the pump 10 alternates between drawing from the first fluid reservoir 58a and the second fluid reservoir 58b. The pump 10 can backprime to remove air from the trap or the proximal tubing and move it into an empty first fluid reservoir 58a or second fluid reservoir 58b. In some implementations, a key, button, or other control (e.g., on an infuser display screen) can be selected to backprime when a delivery is not in progress. When the user selects backprime, for example, this can initiate rapid pumping of fluid from Line A and the common channel to the second fluid reservoir 58b in Line B in an example where the second fluid reservoir 58b is depleted and the pump 10 is drawing from the first fluid reservoir 58a. Similarly, backpriming of the depleted first fluid reservoir can be accomplished by rapid pumping of fluid from Line B and the common channel to the first fluid reservoir 58a in Line A.

Backpriming can occur when the pump controller or processor is configured to actuate the valving and pumping motor to temporarily and for a short period reverse the flow of fluid so that an air bubble or region lacking medical fluid detected in the cassette can be eliminated by returning it to the recently depleted fluid source. Fluid is not drawn in from the patient line during backpriming. In some implementations, for a series of pumping cycles sufficient to eliminate the air bubble or region lacking medical fluid, the outlet valve 231 is closed, the inlet valve 228 is opened and the respective one of the inlet valves 218, 220 that is in fluid communication with the recently depleted fluid source is opened during the pumping strokes and the opposite of inlet valves 218 and 220 is opened during the intake strokes. After a sufficient number of strokes, the air bubble or region lacking medical fluid in the cassette can be returned to the recently depleted fluid source. In some embodiments, (for example, FIG. 2B), backpriming can move fluid towards the depleted Line B line 57b and/or reservoir 58b, with valve 231 and 218 closed and valves 228 and 220 opened during the pump intake cycle and valve 231 and 220 closed and valves 228 and 218 opened during the pump expel cycle. In some embodiments, backpriming can move fluid towards the depleted Line A reservoir 58a, with valve 231 and 220 closed and valves 228 and 218 opened during the pump intake cycle and valves 231 and 218 closed and valves 228 and 220 opened during the pump expel cycle.

Backpriming can be managed by the clinician who manually initiates the backpriming, visually observes air removal from the cassette area up to the Line B container, and then stops the action. In some embodiments, backpriming towards depleted Line A of reservoir 58a can be used. Either backpriming to Line B or to Line A can be clinician-managed or initiated, and/or managed automatically by the pump, to prime lines back to a reservoir spike such as 58a or 58b. Further, either backpriming to line A or line B can be clinician-managed or initiated and managed automatically by the pump to prime lines back to ports such as 253 (FIG. 2C). Backpriming can be done upon system recognition of the accumulated air sensing or pressure sensing, or after each reservoir depletion. Cassette-based pump infusion dedicated consumable sets can include an integrated tubing line terminating with a proximal bag spike (e.g., as the primary Line A), and a direct access port on the cassette (e.g., Line B), which can accommodate a direct connection of a syringe or of secondary tubing connected to a secondary bag. Alternatively, cassette-based infusion pumps can couple with cassettes that include two access ports, which can accommodate direct access for connected syringes or line access to bags. In the case where a line to a reservoir is present, it may be preferable to remove system air by back-priming fluid all the way to the reservoir. Similarly, when a port is available as cassette access, it may be preferable to remove air by back-priming just to the port.

In some implementations, the backpriming is initiated automatically. For example, in some implementations, the control system sends an electrical signal to the pump 10 to automatically backprime when the system alternates between drawing from the first fluid reservoir 58a and the second fluid reservoir 58b, even without detecting an air bubble or region lacking medical fluid in the cassette. In some implementations, the control system sends an electrical signal to the pump 10 to backprime when the first fluid reservoir 58a is depleted, or when the second fluid reservoir 58b is depleted, even without detecting an air bubble or region lacking medical fluid in the cassette.

In some implementations, the backpriming step can happen automatically and very rapidly, without requiring action or approval by a healthcare provider, thereby creating only a very short delay or interruption of fluid flow to the patient (e.g., less than or equal to about 5 seconds or less than or equal to about 10 seconds), permitting substantially continuous infusion to occur even during the transition between the depletion of one fluid source and the start of infusion from another fluid source.

### Terminology and Conclusion

Reference throughout this specification to “some implementations” or “an implementation” means that a particular feature, structure, or characteristic described in connection with the implementation is included in at least some implementations. Thus, appearances of the phrases “in some implementations” or “in an implementation” in various places throughout this specification are not necessarily all referring to the same implementation and may refer to one or more of the same or different implementations. Furthermore, the features, structures, or characteristics may be combined in any suitable manner, as would be apparent to one of ordinary skill in the art from this disclosure, in one or more implementations.

As used in this application, the terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all the elements in the list.

Similarly, it should be appreciated that in this description of implementations, various features are sometimes grouped together in a single implementation, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Rather, inventive aspects lie in a combination of fewer than all features of any single disclosed implementation.

Implementations of the disclosed systems and methods may be used and/or implemented with local and/or remote devices, components, and/or modules. The term “remote” may include devices, components, and/or modules not stored locally, for example, not accessible via a local bus. Thus, a remote device may include a device which is physically located in the same room and connected via a device such as a switch or a local area network. In other situations, a remote device may also be located in a separate geographic area, such as, for example, in a different location, building, city, country, and so forth.

Methods and processes described herein may be embodied in, and partially or fully automated via, software code modules executed by one or more general and/or special purpose computers. The word “module” refers to logic embodied in hardware and/or firmware, or to a collection of software instructions, possibly having entry and exit points, written in a programming language, such as, for example, C or C++. A software module may be compiled and linked into an executable program, installed in a dynamically linked library, or may be written in an interpreted programming language such as, for example, BASIC, Perl, or Python. It will be appreciated that software modules may be callable from other modules or from themselves, and/or may be invoked in response to detected events or interrupts. Software instructions may be embedded in firmware, such as an erasable programmable read-only memory (EPROM). It will be further appreciated that hardware modules may be comprised of connected logic units, such as gates and flip-flops, and/or may be comprised of programmable units, such as programmable gate arrays, application specific integrated circuits, and/or processors. The modules described herein are preferably implemented as software modules, but may be represented in hardware and/or firmware. Moreover, although in some implementations a module may be separately compiled, in other implementations a module may represent a subset of instructions of a separately compiled program, and may not have an interface available to other logical program units.

In certain implementations, code modules may be implemented and/or stored in any type of computer-readable medium or other computer storage device. In some systems, data (and/or metadata) input to the system, data generated by the system, and/or data used by the system can be stored in any type of computer data repository, such as a relational database and/or flat file system. Any of the systems, methods, and processes described herein may include an interface configured to permit interaction with patients, health care practitioners, administrators, other systems, components, programs, and so forth.

A number of applications, publications, and external documents may be incorporated by reference herein. Any conflict or contradiction between a statement in the body text of this specification and a statement in any of the incorporated documents is to be resolved in favor of the statement in the body text.

Terms of equality and inequality (e.g., equal to, less than, greater than) are used herein as commonly used in the field, e.g., accounting for uncertainties present in measurement and control systems. Thus, such terms can be read as approximately equal, approximate less than, and/or approximately greater than. In other aspects of the invention, an acceptable threshold of deviation or hysteresis can be established by the pump manufacturer, the editor of the drug library, or the user of a pump.

While the implementations of the invention disclosed herein are presently considered to be preferred, various changes and modifications can be made without departing from the scope of the invention. Although described in the illustrative context of certain preferred implementations and examples, it will be understood by those skilled in the art that the disclosure extends beyond the specifically described implementations to other alternative implementations and/or uses and obvious modifications and equivalents. Thus, it is intended that the scope of the claims which follow should not be limited by the particular implementations described above. The scope of the invention is indicated in the appended claims, and all changes that come within the meaning and range of equivalents are intended to be embraced therein.

The following is claimed:

1. A control system for controlling operation of an infusion pump of an infusion pump system, the infusion pump system comprising a first fluid reservoir and a first supply line, a second fluid reservoir and a second supply line, a common channel in selective fluidic communication with the first supply line and the second supply line, and an infusion pump, wherein the infusion pump is operable to drive liquid through the common channel to a patient, the control system comprising:
one or more hardware processors; and
a memory storing executable instructions that when executed by the one or more hardware processors, configure the infusion pump to:
draw liquid from the first fluid reservoir and first supply line;
automatically discontinue drawing liquid from the first fluid reservoir and first supply line and begin drawing fluid from the second fluid reservoir and second supply line upon receiving an indication that the first fluid reservoir is in a depletion zone and
automatically discontinue drawing fluid from the second fluid reservoir and second supply line and begin drawing fluid from the first fluid reservoir and first fluid line upon receiving an indication that the second fluid reservoir is in the depletion zone,
wherein the control system is configured to deliver to the patient substantially continuously fluid drawn including during transitions between the first fluid reservoir and the second fluid reservoir.

2. The control system of claim 1, wherein the infusion pump is configured to automatically draw fluid from at least the first supply line or at least the second supply line until the infusion pump receives a signal from an air-in-line sensor detecting air or a lack of liquid within a region of the cassette inserted into the pump.

3. The control system of claim 1, wherein each indication is based on a period of time for at least one of the first fluid reservoir and the second fluid reservoir to enter the depletion zone based upon a container volume.

4. The control system of claim 3, wherein the container volume is determined by an electronically readable data source on the first or second fluid reservoir.

5. The control system of claim 1, wherein each indication is based on a signal from a pressure sensor in communication with a respective reservoir and supply line.

6. The control system of claim 5, wherein the indication that the first fluid reservoir and first supply line are depleted and the indication that the second fluid reservoir and the second supply line are depleted is an upstream pressure measured by the at least one pressure sensor.

7. The control system of claim 1, wherein the infusion pump draws fluid from the first fluid reservoir only upon receiving an indication of first fluid reservoir fluid availability, and wherein the infusion pump draws from the second fluid reservoir only upon receiving an indication of second fluid reservoir fluid availability.

8. The control system of claim 1, wherein the operation is configured to draw liquid from a first or second reservoir that is a bag.

9. The control system of claim 1, wherein the operation is configured to draw liquid from a first or second reservoir that is a syringe.

10. The control system of claim 1, wherein the infusion pump is configured to automatically draw fluid from at least one of the first fluid reservoir and the second fluid reservoir for a predetermined period of time.

11. The control system of claim 1, wherein the infusion pump is further configured to back prime fluid from the common channel into the first fluid reservoir when the first fluid reservoir is depleted, and
wherein discontinuing drawing fluid from the first fluid reservoir further comprises back priming fluid from the common channel into the first fluid reservoir.

12. The control system of claim 1, wherein the infusion pump is further configured to back prime fluid from the common channel into the second fluid reservoir when the second fluid reservoir is depleted, and
wherein discontinuing drawing fluid from the second fluid reservoir further comprises back priming fluid from the common channel into the second fluid reservoir.

13. A method for controlling operation of an infusion pump of an infusion pump system, the infusion pump system comprising a first fluid reservoir, a second fluid reservoir, a common channel in selective fluidic communication with the first fluid reservoir and the second fluid reservoir, and an infusion pump, wherein the infusion pump is operable to drive fluid through the common channel, the method comprising:
drawing fluid from the first fluid reservoir through the common channel;
automatically discontinuing drawing fluid from the first fluid reservoir and drawing fluid from the second fluid reservoir through the common channel upon receiving an indication the first fluid reservoir is depleted; and
automatically discontinuing drawing fluid from the second fluid reservoir and drawing fluid from the first fluid reservoir through the common channel upon receiving an indication that the second fluid reservoir is depleted,
wherein fluid drawn from the first fluid reservoir through the common channel is substantially successively continuous with fluid drawn from the second fluid reservoir through the common channel.

14. The method of claim 13, wherein the indication that the first fluid reservoir is depleted and the indication that the second fluid reservoir is depleted is an upstream pressure measured by the at least one pressure sensor.

15. The method of claim 13, wherein the infusion pump draws fluid from the first fluid reservoir only upon receiving an indication of first fluid reservoir fluid availability, and wherein the infusion pump draws from the second fluid reservoir only upon receiving an indication of second fluid reservoir fluid availability.

16. The method of claim 15, wherein the indication is a threshold weight of at least one of the first fluid reservoir and the second fluid reservoir.

17. The method of claim 13, wherein the infusion pump is configured to automatically draw fluid from at least one of the first fluid reservoir and supply line and the second fluid reservoir and supply line for a predetermined period of time.

18. The method of claim 13, wherein the infusion pump is further configured to back prime fluid from the common channel into the first fluid reservoir when the first fluid reservoir is depleted, and
wherein discontinuing drawing fluid from the first fluid reservoir further comprises back priming fluid from the common channel into the first fluid reservoir.

19. The method of claim 13, wherein the infusion pump is further configured to back prime fluid from the common channel into the second fluid reservoir when the second fluid reservoir is depleted, and
wherein discontinuing drawing fluid from the second fluid reservoir further comprises back priming fluid from the common channel into the second fluid reservoir.

20. A control system for controlling operation of an infusion pump of an infusion pump system, the infusion pump system comprising a first fluid reservoir, a second fluid reservoir, a common channel in selective fluidic communication with the first fluid reservoir and the second fluid reservoir, and an infusion pump, wherein the infusion pump is operable to drive fluid through the common channel, the control system comprising:
one or more hardware processors; and
a memory storing executable instructions that when executed by the one or more hardware processors, configure the infusion pump to:
draw fluid from the first fluid reservoir through the common channel;
automatically discontinue drawing fluid from the first fluid reservoir and draw fluid from the second fluid reservoir through the common channel upon receiving an indication that the first fluid reservoir is depleted; and
automatically discontinue drawing fluid from the second fluid reservoir and draw fluid from the first fluid reservoir through the common channel upon receiving instructions to draw fluid from the first fluid reservoir,
wherein fluid drawn from the first fluid reservoir through the common channel is substantially successively continuous with fluid drawn from the second fluid reservoir through the common channel.

21. The control system of claim 20, wherein the infusion pump is further configured to back prime fluid from the common channel into the first fluid reservoir when the first fluid reservoir is depleted, and
wherein discontinuing drawing fluid from the first fluid reservoir further comprises back priming fluid from the common channel into the first fluid reservoir.

22. The control system of claim 20, wherein the infusion pump is further configured to back prime fluid from the common channel into the second fluid reservoir when the second fluid reservoir is depleted, and
wherein discontinuing drawing fluid from the second fluid reservoir further comprises back priming fluid from the common channel into the second fluid reservoir.
